(19) United States
(12) Patent Application Publication
(10) Pub. No.: US 2024/0131113 A1
(43) Pub. Date: Apr. 25, 2024

(54) PROTEIN-BASED THERAPIES FOR OCULAR CONDITIONS

(71) Applicants: The Regents of the University of Colorado, A Body Corporate, Denver, CO (US); The University of North Texas Health Science Center at Fort Worth, Forth Worth, TX (US)

(72) Inventors: Ram H. NAGARAJ, Aurora, CO (US); Rooban B. NAHOMI, Aurora, CO (US); Dorota L. STANKOWSKA, Forth Worth, TX (US)

(21) Appl. No.: 18/547,382

(22) PCT Filed: Feb. 22, 2022
(86) PCT No.: PCT/US2022/017278

§ 371 Date: Aug. 21, 2023

Related U.S. Application Data
(60) Provisional application No. 63/152,128, filed on Feb. 22, 2021.
(60) Provisional application No. 63/273,643, filed on Oct. 29, 2021.

(51) Int. Cl. A61K 38/17; A61K 9/00; A61K 47/64; A61P 27/06; C07K 7/06; C07K 14/47

(52) U.S. Cl. CPC A61K 38/1709; A61K 9/0019; A61K 9/0048; A61K 47/64; A61P 27/06; C07K 7/06; C07K 14/47

(57) ABSTRACT

Peptide-based therapies for a retinal disease, injury, or condition in a subject involve administering to the subject a pharmaceutical composition containing at least one peptide derived from a heat shock protein, including Hsp20 and αB-crystallin. The administered peptides may be acetylated and/or conjugated to a cell-penetrating peptide. Administration of the peptides may reduce or prevent the loss of at least one retinal cell type, including retinal ganglion cells and retinal endothelial cells. The loss of such cells causes retinal damage and loss of eyesight in patients afflicted with an ocular condition. The pharmaceutical compositions may be administered intravitreally using an administration device. A single injection may be therapeutically sufficient for treating various ocular conditions.

### CROSS-REFERENCE TO RELATED APPLICATIONS

This application claims priority to U.S. Provisional Patent Application No. 63/152,128, filed Feb. 22, 2021, entitled “Protein-Based Therapies for Ocular Conditions,” and U.S. Provisional Patent Application No. 63/273,643, filed Oct. 29, 2021, entitled “Protein-Based Therapies for Ocular Conditions,” each of which is incorporated herein by reference in the entirety and for all purposes.

### STATEMENT OF GOVERNMENT INTEREST

This invention was made with government support from the National Institute of Health under grant number 5R01EY028179-02. The government has certain rights in the invention.

### TECHNICAL FIELD

The present disclosure relates generally to compositions, systems, and methods for treating retinal damage caused by injury or disease. Specific implementations involve the delivery of at least one heat shock peptide, or portion thereof, to the retinal cells of a subject afflicted with, or at risk of developing, ocular damage.

### BACKGROUND

Glaucoma affects nearly 75 million people worldwide, and approximately 8 million people are blind from the disease. Nearly 3 million people are afflicted with glaucoma in the United States alone, and this number is expected to more than double by 2050. Because glaucoma-associated vision loss is often attributed largely to elevated pressure inside the eye, known as intraocular pressure, the conventional first line of glaucoma treatment usually involves topical application of drugs formulated to lower intraocular pressure. Even if this approach successfully lowers the pressure, however, many patients still go blind because of axonal degeneration and the continued death of cells in the retina, known as retinal ganglion cells (“RGCs”). The diversity of factors contributing to axonal degeneration and RGC death, both individually and especially in combination, make glaucoma and other ocular conditions difficult to treat. Accordingly, safe and effective methods of combating RGC death and axonal degeneration are needed.

### SUMMARY

The present disclosure includes novel peptide-based therapies for various ocular conditions, including glaucoma. Embodiments include peptides derived from heat shock proteins (“HSPs”). The disclosed HSP peptides may be injected intravitreally to provide a targeted, localized effect in the eye(s) of a subject. Successful treatment, prevention, and/or alleviation of at least one symptom of an ocular condition may be achieved via administration of one or more of the disclosed HSP peptides, which may be conjugated with a cell-penetrating peptide (“CPP”) to increase cellular penetration and effect. As illustrated by the experimental data summarized herein, the disclosed HSP peptides, pharmaceutical compositions, and associated therapies may prevent or treat retinal damage by substantially blocking, slowing and/or reducing intraocular tension, RGC death, retinal endothelial cell death, inflammatory cytokine production, axonal degeneration, and/or retinal capillary degeneration.

In accordance with specific embodiments of the present disclosure, a method of treating, reducing the risk of, preventing, and/or alleviating at least one symptom of a retinal disease, injury, or condition in a subject may involve intravitreally administering to the subject a therapeutically effective amount of a composition comprising at least one polypeptide derived from a biologically active heat shock protein, such as Hsp20. The at least one polypeptide may have an amino acid sequence at least 90% identical to G⁷³HFSVLLDVKHFSPEEIAVK⁹¹.

In some embodiments of the method, the polypeptide may be acetylated. In some embodiments of the method, the polypeptide may have an amino acid sequence at least 90% identical to G⁷³HFSVLLDVK(acetyl)HFSPEEIAVK⁹¹. In some embodiments of the method, the polypeptide may exhibit molecular chaperone activity. In some embodiments of the method, the composition may be administered during or after an ocular surgical procedure. In some embodiments, the retinal disease, injury, or condition may be glaucoma. In some embodiments of the method, the retinal disease, injury, or condition may be selected from the group consisting of: macular degeneration, diabetic retinopathy, retinal detachment, and retinitis pigmentosa. In some embodiments of the method, the retinal disease, injury, or condition may be caused by excitotoxic damage, physical damage, chemical damage, neurotrophic factor deprivation, oxidative stress, inflammation, mitochondrial dysfunction, axonal transport failure, or combinations thereof. In some embodiments of the method, the retinal disease, injury, or condition may include a loss of human retinal ganglion cells. In some embodiments of the method, the retinal disease, injury, or condition may include ocular hypertension. In some embodiments of the method, the retinal disease, injury, or condition may include optic nerve degeneration. In some embodiments of the method, the retinal disease, injury, or condition may include pathological apoptosis and/or protein aggregation.

In accordance with specific embodiments of the present disclosure, a system for treating, reducing the risk of, preventing, or alleviating at least one symptom of a retinal disease, injury, or condition in a subject may include a therapeutically effective amount of a composition comprising at least one polypeptide derived from a biologically active heat shock protein, such as Hsp20. The polypeptide may have an amino acid sequence at least 90% identical to G⁷³HFSVLLDVKHFSPEEIAVK⁹¹. The system may also include an intravitreal injection device configured to administer the composition to the subject.

In some embodiments of the system, the polypeptide may be acetylated. In some embodiments of the system, the polypeptide may have an amino acid sequence at least 90% identical to G⁷³HFSVLLDVK(acetyl)HFSPEEIAVK⁹¹. In some embodiments of the system, the intravitreal injection device may be a tuberculin, Hamilton, or Tribofilm Staclear-type syringe. In some embodiments of the system, the retinal disease, injury, or condition may be selected from the group consisting of: glaucoma, macular degeneration, diabetic retinopathy, retinal detachment, retinitis pigmentosa, retinal ganglion cell loss, retinal endothelial cell loss, retinal capillary cell loss, ocular hypertension, optic nerve degeneration, pathological apoptosis, and protein aggregation.

In accordance with specific embodiments of the present disclosure, a pharmaceutical composition may comprise at least one polypeptide derived from Hsp20. The polypeptide may have an amino acid sequence at least 90% identical to G⁷³HFSVLLDVKHFSPEEIAVK⁹¹. The pharmaceutical composition may also include a pharmaceutically acceptable carrier. The pharmaceutical composition may be formulated for treating, reducing the risk of, preventing, or alleviating at least one symptom of a retinal disease, injury, or condition in a subject. The pharmaceutical composition may be formulated for intravitreal administration.

In some embodiments of the composition, the polypeptide may be acetylated. In some embodiments of the composition, the polypeptide may have an amino acid sequence at least 90% identical to G⁷³HFSVLLDVK(acetyl)HFSPEEIAVK⁹¹. In some embodiments of the composition, the retinal disease, injury, or condition may be selected from the group consisting of: glaucoma, macular degeneration, diabetic retinopathy, retinal detachment, retinitis pigmentosa, retinal ganglion cell loss, retinal endothelial cell loss, retinal capillary cell loss, ocular hypertension, optic nerve degeneration, pathological apoptosis, and protein aggregation.

In accordance with specific embodiments of the present disclosure, a pharmaceutical composition that includes at least one polypeptide derived from a biologically active heat shock protein may be used in the manufacture of a medicament for treating, reducing the risk of, preventing, or alleviating at least one symptom of a retinal disease, injury, or condition in a subject. The heat shock protein may be Hsp20. The polypeptide may have an amino acid sequence at least 90% identical to G⁷³HFSVLLDVKHFSPEEIAVK⁹¹, and the pharmaceutical composition may be formulated for intravitreal administration.

In some manufacturing embodiments, the polypeptide may be acetylated. In some manufacturing embodiments, the polypeptide may have an amino acid sequence at least 90% identical to G⁷³HFSVLLDVK(acetyl)HFSPEEIAVK⁹¹. In some manufacturing embodiments, the retinal disease, injury, or condition may be selected from the group consisting of: glaucoma, macular degeneration, diabetic retinopathy, retinal detachment, retinitis pigmentosa, retinal ganglion cell loss, retinal endothelial cell loss, retinal capillary cell loss, ocular hypertension, optic nerve degeneration, pathological apoptosis, and protein aggregation.

In accordance with specific embodiments of the present disclosure, a method of treating, reducing the risk of, preventing, and/or alleviating at least one symptom of a retinal disease, injury, or condition in a subject may involve intravitreally administering to the subject a therapeutically effective amount of a composition comprising at least one polypeptide derived from a biologically active heat shock protein, such as αB-crystallin. The polypeptide may have an amino acid sequence at least 90% identical to ⁷³DRF SVNLDVKHFSPEELKVKV⁹³.

In some embodiments of the method, the polypeptide may be conjugated with a cell-penetrating peptide. In some embodiments of the method, the cell-penetrating peptide may have an amino acid sequence at least 80% identical to VPTLK. In some embodiments of the method, the composition may be administered during or after an ocular surgical procedure.

In some embodiments of the method, the retinal disease, injury, or condition may be glaucoma. In some embodiments of the method, the retinal disease, injury, or condition may comprise a loss of human retinal ganglion cells. In some embodiments of the method, the retinal disease, injury, or condition may comprise a loss of human retinal ganglion cell function. In some embodiments of the method, the retinal disease, injury, or condition may be caused by physical damage, chemical damage, neurotrophic factor deprivation, or combinations thereof. In some embodiments of the method, the retinal disease, injury, or condition may comprise ocular hypertension. In some embodiments of the method, the retinal disease, injury, or condition may comprise optic nerve degeneration.

In accordance with specific embodiments of the present disclosure, a system for treating, reducing the risk of, preventing, or alleviating at least one symptom of a retinal disease, injury, or condition in a subject may include a therapeutically effective amount of a composition comprising at least one polypeptide derived from a biologically active heat shock protein, such as αB-crystallin. The polypeptide may have an amino acid sequence at least 90% identical to ⁷³DRFSVNLDVKHFSPEELKVKV⁹³. The system may also include an intravitreal injection device configured to administer the composition to the subject.

In some embodiments of the system, the polypeptide may be conjugated with a cell-penetrating peptide. In some embodiments of the system, the cell-penetrating peptide may have an amino acid sequence at least 80% identical to VPTLK. In some embodiments of the system, the intravitreal injection device may be a tuberculin syringe. In some embodiments of the system, the retinal disease, injury, or condition may be selected from the group consisting of glaucoma, retinal ganglion cell loss, retinal ganglion cell functional decline, retinal endothelial cell loss, ocular hypertension, and optic nerve degeneration.

In accordance with specific embodiments of the present disclosure, a pharmaceutical composition may comprise at least one polypeptide derived from αB-crystallin. The polypeptide may have an amino acid sequence at least 90% identical to ⁷³DRFSVNLDVKHFSPEELKVKV⁹³. The pharmaceutical composition may also include a pharmaceutically acceptable carrier. The pharmaceutical composition may be formulated for treating, reducing the risk of, preventing, or alleviating at least one symptom of a retinal disease, injury, or condition in a subject. The pharmaceutical composition may be formulated for intravitreal administration.

In some embodiments of the pharmaceutical composition, the polypeptide may be conjugated with a cell-penetrating peptide, which may have an amino acid sequence at least 80% identical to VPTLK. In some embodiments of the pharmaceutical composition, the retinal disease, injury, or condition may be selected from the group consisting of glaucoma, retinal ganglion cell loss, retinal ganglion cell functional decline, retinal endothelial cell loss, ocular hypertension, and optic nerve degeneration.

In accordance with specific embodiments of the present disclosure, a pharmaceutical composition that includes at least one polypeptide derived from a biologically active heat shock protein may be used in the manufacture of a medicament for treating, reducing the risk of, preventing, or alleviating at least one symptom of a retinal disease, injury, or condition in a subject. The heat shock protein may comprise αB-crystallin. The polypeptide may have an amino acid sequence at least 90% identical to ⁷³DRFSVNLDVKHFSPEELKVK⁹². The pharmaceutical composition may be formulated for intravitreal administration. In some manufacturing embodiments, the polypeptide may be conjugated with a cell-penetrating peptide, which may have an amino acid sequence at least 80% identical to VPTLK. In some manufacturing embodiments, the retinal disease, injury, or condition may be selected from the group consisting of glaucoma, retinal ganglion cell loss, retinal ganglion cell functional decline, retinal endothelial cell loss, ocular hypertension, and optic nerve degeneration.

This Summary is neither intended as, nor should it be construed as, being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and Detailed Description, and no limitation as to the scope of the present disclosure is intended by either the inclusion or non-inclusion of elements, components, etc. in this Summary. Features from any of the disclosed embodiments may be used in combination with one another, without limitation. In addition, other features and advantages of the present disclosure will become apparent to those of ordinary skill in the art through consideration of the following Detailed Description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1A is a line graph showing the effects of microbead injection on intraocular pressure according to embodiments disclosed herein.

FIG. 1B is a bar graph showing the effects of intravitreal HSP peptide administration on RGC death in a microbead-based mouse model of ocular hypertension according to embodiments disclosed herein.

FIG. 1C is a confocal microscopy image showing the effects of intravitreal HSP peptide administration on RGCs derived from healthy mice, or mice afflicted with ocular hypertension, using Brna3- and β-III-tubulin immunostaining according to embodiments disclosed herein.

FIG. 2A is a line graph showing the effects of silicone oil injection on intraocular pressure according to embodiments disclosed herein.

FIG. 2B is a bar graph showing the effects of intravitreal HSP peptide administration on RGC death in a silicone oil-based mouse model of ocular hypertension according to embodiments disclosed herein.

FIG. 2C is a confocal microscopy image showing the effects of intravitreal HSP peptide administration on RGCs derived from healthy mice, or mice afflicted with ocular hypertension, using Brna3-immunostaining according to embodiments disclosed herein.

FIG. 3A is a bar graph and corresponding Western blot showing the effects of pro-inflammatory cytokine exposure on the survival of human retinal endothelial cells according to embodiments disclosed herein.

FIG. 3B is a bar graph and corresponding Western blot showing the effects of HSP peptide administration on the survival of human retinal endothelial cells after pro-inflammatory cytokine exposure according to embodiments disclosed herein.

FIG. 3C is a bar graph and corresponding Western blot showing HSP peptide levels in human retinal endothelial cells after administration of the peptide according to embodiments disclosed herein.

FIG. 4A is a confocal microscopy image showing the presence of an HSP peptide tagged with Cy5 dye in the retinas of mice intravitreally injected with the HSP peptide according to embodiments disclosed herein.

FIG. 4B is a bar graph showing the fluorescence levels detected in the retinal cells depicted in FIG. 4A.

FIG. 4C is a confocal microscopy image showing the presence of the HSP peptide of FIG. 4A in the RGCs of mice intravitreally injected with the HSP peptide according to embodiments disclosed herein.

FIG. SA is a microscopy image showing the effects of HSP peptide administration on retinal capillary cell degeneration after ocular injury according to embodiments disclosed herein.

FIG. 5B is a bar graph showing the number of acellular retinal cells detected in FIG. 5A.

FIG. 6 comprises three bar graphs showing the effects of HSP peptide administration on inflammatory cytokine expression in the retina following ocular injury according to embodiments disclosed herein.

FIG. 7 is a bar graph and corresponding Western blot showing the penetration of peptain-1 (P1) conjugated with a cell-penetrating peptide (CPP) into retinal endothelial cells according to embodiments disclosed herein.

FIG. 8A is a Cytation5 microscopy image panel showing the impact of peptain-1 conjugated with a cell-penetrating peptide (P1-CPP) on the protection of rat primary RGCs after trophic factor deprivation according to embodiments disclosed herein.

FIG. 8B is a bar graph showing the quantitative effect of peptain-1 conjugated with the cell-penetrating peptide (P1-CPP) on the protection of rat primary RGCs after trophic factor deprivation according to embodiments disclosed herein.

FIG. 9A is a Cytation5 microscopy image panel showing the impact of peptain-1 conjugated with a cell-penetrating peptide (P1-CPP) on the protection of rat primary RGCs after endothelin-3-induced death according to embodiments disclosed herein.

FIG. 9B is a bar graph showing the quantitative effects of peptain-1 conjugated with a cell-penetrating peptide (P1-CPP) on the protection of rat primary RGCs after endothelin-3-induced death according to embodiments disclosed herein.

FIG. 10A is a Cytation5 microscopy image panel showing the impact of peptain-1 conjugated with a cell-penetrating peptide (P1-CPP) on the protection of rat RGCs in the peripheral retina following 6 weeks of intraocular pressure elevation using Morrison's model of ocular hypertension detected by immunolabeling with retinal ganglion cell marker Brna3 according to embodiments disclosed herein.

FIG. 10B is a bar graph showing the quantitative effect of peptain-1 conjugated with a cell-penetrating peptide (P1-CPP) on RGC protection in the peripheral retina following 6 weeks of intraocular pressure elevation in Brown Norway rats according to embodiments disclosed herein.

FIG. 10C is a bar graph showing the quantitative effect of peptain-1 conjugated with a cell-penetrating peptide (P1-CPP) on the protection of RGCs in the mid-peripheral retina following 6 weeks of intraocular pressure elevation in Brown Norway rats according to embodiments disclosed herein.

FIG. 10D is a line graph showing the intraocular pressure profiles measured in IOP-elevated and contralateral eyes to verify the elevated intraocular pressure conditions reflected in FIGS. 10A-10C according to embodiments disclosed herein.

FIG. 11 is a light microscopy image panel showing the impact of peptain-1 conjugated with a cell-penetrating peptide (P1-CPP) on the protection of optic nerve axons after 6 weeks of intraocular pressure elevation in Brown Norway rats according to embodiments disclosed herein.

FIG. 12A is a bar graph showing the impact of peptain-1 conjugated with a cell-penetrating peptide (P1-CPP) on RGC function after 6 weeks of intraocular pressure elevation in Brown Norway rats according to embodiments disclosed herein.

FIG. 12B is a collection of pattern electroretinogram (“PERG”) traces corresponding to the bar graph of FIG. 12A.

FIG. 13A is a bar graph showing the quantitative effect of peptain-1 conjugated with a cell-penetrating peptide (P1-CPP) on the protection of human RGCs following 7 days of ex vivo culture in a human retinal explant model of RGC death according to embodiments disclosed herein.

FIG. 13B is a confocal microscopy image panel showing the impact of peptain-1 conjugated with a cell-penetrating peptide (P1-CPP) on the protection of the human RGCs represented in FIG. 13A.

FIG. 14A is a bar graph representing RNA sequencing results showing significantly unregulated and downregulated canonical pathways in rat RGCs isolated following 2 weeks of IOP elevation and P1-CPP treatment compared to 2 weeks of IOP elevation and vehicle treatment according to embodiments disclosed herein.

FIG. 14B is a heat map analysis of the top 12 differentially expressed genes in the pro-survival CREB pathway of RGCs isolated from IOP-elevated, P1-CPP treated rats compared to RGCs isolated from IOP-elevated vehicle control rats according to embodiments disclosed herein.

FIG. 14C is a bar graph showing the relative Creb 1 gene expression fold change in RGCs isolated from IOP-elevated, P1-CPP treated rats compared to IOP-elevated vehicle treated control rats according to embodiments disclosed herein.

### DETAILED DESCRIPTION

This disclosure relates to compositions, methods, and systems for treating, reducing the risk of, preventing, and/or alleviating at least one symptom of a retinal disease, injury, or condition, including glaucoma and associated ocular damage. Embodiments involve reducing or preventing retinal cell death via peptide delivery approaches that involve administering a pharmaceutical composition containing at least one HSP peptide, such as a peptide derived from Hsp20 or αB-crystallin. In some embodiments, the HSP peptide may be conjugated with a CPP, which may increase the penetration of the HSP peptide into the targeted cells, e.g., RGCs, thereby also increasing the desired effects of the HSP peptide. To deliver exogenous HSP peptides to retinal cells, the pharmaceutical composition may be administered intravitreally. The pharmaceutical composition, which may also include an acceptable carrier and/or excipient, may be administered one or more times before and/or after a subject is diagnosed with an ocular condition, such as glaucoma, or after a subject sustains an eye injury. Administration of the pharmaceutical composition in the manner disclosed, e.g., directly into one or both eyes of a subject, may increase the levels of anti-apoptotic HSP peptides in the eye, thereby significantly inhibiting retinal cell death, lowering intraocular pressure, and/or reducing axonal degeneration that would otherwise occur after the injury or onset of an ocular condition. While not limited to any particular theory, administration of the pharmaceutical composition may upregulate one or more components of the pro-survival CREB signaling pathway. These benefits may prevent, ameliorate, and/or impede retinal damage in a safe, effective manner not previously contemplated in the field of ocular therapy.

Unless defined otherwise below, technical and scientific terms used herein have the same meaning as commonly understood by one of ordinary skill in the art to which this invention pertains. For the purposes of the present invention, the following terms are defined for clarity.

As used herein, “HSPs” are stress proteins each having a crystallin core domain ranging from about 80 to about 100 amino acid residues. Among additional physiological functions, HSPs may exhibit anti-apoptotic and molecular chaperone activity within the cells in which they are present. HSPs may be divided categorically into small HSP peptides (˜12-43 kDa) and large HSP peptides (˜100-110 kDa). Examples of small HSP peptides include α-crystallin (which is comprised of two subunits, αA andα), Hsp20, and Hsp27.

The “HSP peptides” referenced herein are polypeptides derived from HSPs, such as Hsp20 and αB-crystallin. The terms “peptide” or “polypeptide” may be used interchangeably herein. The number of amino acids constituting each HSP peptide may vary, ranging in various embodiments from about two to about 50, about six to about 40, about ten to about 30, about 12 to about 28, about 14 to about 26, about 16 to about 24, about 18 to about 22, about 19, about 20, or about 21 amino acids. The HSP peptides may each constitute a portion of the crystallin core domain of the full-length protein from which they are derived. For example, the HSP peptides may include about 20-25% of the amino acids present in the crystallin core domain of the HSPs from which they are derived. Despite their smaller size, the HSP peptides may retain the molecular chaperone and anti-apoptotic properties of the corresponding full-sized crystallin core domains. Each amino group of each amino acid is linked to the carboxyl group of another amino acid via a peptide bond. The HSP peptides disclosed herein may be derived from directly from naturally occurring HSPs of human or non-human origin by enzymatic and/or chemical cleavage. Alternatively, the HSP peptides may be prepared by a peptide synthesis technique. The resulting HSP peptides may be present in various forms, such as a chain of amino acids with or without a degree of three-dimensional folding. Like the HSPs from which they are derived, the disclosed HSP peptides may be cell-permeable, may inhibit protein aggregation and apoptosis, and may exhibit robust molecular chaperone activity. Accordingly, the peptides may be effective to prevent or treat diseases or conditions in which protein aggregation and apoptosis are implicated.

The HSP peptides may be acetylated in some embodiments and not acetylated in other embodiments. An “acetylated” peptide is a peptide that includes at least one acetylated amino acid, e.g., lysine. Chemical acetylation may be performed in some examples to generate acetylated HSP peptides prior to their inclusion in a pharmaceutical composition. The acetylated peptides disclosed herein may be less susceptible to proteolytic enzymes, thereby rendering them more stable in vivo.

As used herein, “subject” means a human or other mammal. Non-human subjects may include, but are not limited to, various mammals such as domestic pets and/or livestock, for example. A subject may be considered in need of treatment. The disclosed compositions, methods, and systems may be effective to treat healthy human subjects, patients diagnosed with glaucoma or diabetic retinopathy, patients diagnosed with one or more other ocular diseases, patients suffering from various eye injuries, diabetic patients, or patients experiencing loss of eyesight.

As used herein, an “ocular condition” encompasses all diseases or conditions related to the eye, including those that negatively affect one or both eyes of a subject. Ocular diseases, injuries, and conditions targeted by the therapeutic methods disclosed herein may damage retinal tissue specifically, including RGCs, retinal endothelial cells, and retinal capillaries. Non-limiting examples of ocular conditions contemplated herein may include glaucoma, macular degeneration, cataract formation, diabetic eye disease, diabetic retinopathy, retinal detachment, retinitis pigmentosa, RGC death, elevated intraocular pressure, ocular hypertension, axonal degeneration, excitotoxic damage, physical damage (e.g., ischemia and/or reperfusion), chemical damage, neurotrophic factor deprivation, oxidative stress, inflammation, mitochondrial dysfunction, axonal transport failure, or combinations thereof.

As used herein “glaucoma” refers to a disease characterized by the permanent loss of visual function due to irreversible damage to the optic nerve. The two main types of glaucoma are primary open angle glaucoma and angle closure glaucoma, one or both of which may be treated according to embodiments described herein.

As used herein, the term “intraocular pressure” refers to the pressure of the fluid inside the eye. The intraocular pressure of a normal human eye typically ranges from about 10 to about 21 mm Hg. “Elevated” intraocular pressure or “ocular hypertension” is conventionally considered to be greater than or equal to about 21 mm Hg. Elevated intraocular pressure may be a risk factor for the development of glaucoma.

Treating retinal damage, as contemplated herein, encompasses treating, reducing the risk of, preventing, or alleviating at least one symptom of retinal damage caused by or associated with a disease, injury, or other condition. Accordingly, “treating,” “treatment,” or “alleviation” refers to both therapeutic treatment and prophylactic or preventative measures, wherein the object is to prevent or slow down (lessen) the targeted pathological condition and/or symptom. Those in need of treatment include those already diagnosed with the condition, as well as those prone to contracting or developing the condition. A subject is successfully “treated” for retinal damage if, after receiving a therapeutically effective amount of a pharmaceutical composition according to methods of this disclosure, the subject shows observable and/or measurable reduction in, or absence of, one or more of eyesight impairment, eyesight loss, eyesight abnormalities, axonal degeneration, RGC death, retinal endothelial cell death, and/or retinal capillary degeneration. The terms “treat” or “treating” are used consistently herein for ease of illustration only, and thus should not be construed as limiting.

“Reducing,” “reduce,” or “reduction” means decreasing the severity, scope, frequency, or length of retinal damage.

An “effective amount” of a composition containing an HSP peptide or combination of HSP peptides is an amount sufficient to carry out a specifically stated purpose, and may be determined empirically and in a routine manner, in relation to the stated purpose. For example, an “effective amount” as used herein may be defined as an amount of an HSP peptide that, upon administration to a subject, will restore or exceed natural HSP levels in the RGCs, retinal endothelial cells, and/or retinal capillary cells of the subject. The term “therapeutically effective amount” refers to an amount of a composition containing an HSP peptide that will detectably and repeatedly treat, reduce the risk of, prevent, or alleviate at least one symptom of a retinal disease, injury, or condition in a subject. This includes, but is not limited to, a reduction in the frequency or severity of the signs or symptoms of a disease, such as elevated intraocular pressure, RGC death, retinal endothelial cell death, retinal capillary degeneration, vision loss, RGC soma degeneration, and/or RGC axonal degeneration. Such improvements may be considered relative to an eye of a subject not administered a disclosed pharmaceutical composition according to the methods disclosed herein. One skilled in the art understands that a treatment may improve a disease condition, but may not be a complete cure for the disease. For example, successful treatment of a patient with glaucoma may be evidenced by no further progression of visual field loss in the affected eye, or a slowing of the rate of progression of visual field loss in the affected eye.

“Administration of” and “administering a” compound, composition, or agent should be understood to mean providing a compound, composition, or agent, a prodrug of a compound, composition, or agent, or a pharmaceutical composition as described herein. The compound, agent or composition may be provided or administered by another person to the subject (e.g., intravitreally) or it may be self-administered by the subject.

“Pharmaceutical compositions” or “pharmaceutical formulations” are compositions that include an amount (for example, a unit dosage) of one or more of the disclosed compounds, e.g., acetylated or non-acetylated HSP peptides optionally conjugated with a CPP, together with one or more non-toxic pharmaceutically acceptable additives, including carriers, diluents, and/or adjuvants, and optionally other biologically active ingredients. Such pharmaceutical compositions may be prepared by standard pharmaceutical formulation techniques such as those disclosed in Remington's Pharmaceutical Sciences, Mack Publishing Co., Easton, Pa. (19th Edition).

As used herein, a “pharmaceutically acceptable excipient” or a “pharmaceutically acceptable carrier” means a pharmaceutically acceptable material, composition, or vehicle that contributes to the desired form or consistency of the pharmaceutical composition. Each excipient or carrier must be compatible with other ingredients of the pharmaceutical composition when comingled such that interactions which would substantially reduce the efficacy of the compositions of this disclosure when administered to a subject and interactions which would result in pharmaceutical compositions that are not pharmaceutically acceptable are avoided. In addition, each excipient or carrier must be of sufficiently high purity to render it pharmaceutically acceptable. Non-limiting examples of pharmaceutically acceptable carriers may include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methylhydroxybenzoate, propylhydroxybenzoate, talc, magnesium stearate, mineral oil or the like. In addition or alternatively, a carrier may include comprise a lubricant, a wetting agent, a flavor, an emulsifier, a suspending agent, a preservative, or the like.

As used herein, “wild-type” refers to the naturally occurring protein or portion thereof, as it normally exists in vivo.

The agents, compounds, compositions, antibodies, etc. used in the implementations described herein are considered to be purified and/or isolated prior to use. Purified materials are typically “substantially pure,” meaning that an HSP peptide or other molecule has been separated from the components that naturally accompany it. For example, an HSP peptide may be considered substantially pure when it is at least 50%, 55%, 60%, 65%, 70%, 75%, 80%, 85%, 90%, 95%, 98%, 98%, or 99% free (by weight) from the proteins and other organic molecules with which it is associated naturally.

As used herein, the terms “identity” or “similarity” denote relationships between two or more polypeptide sequences or their underlying nucleic acid sequences, as determined by comparing the sequences. In the art, “identity” also means the degree of sequence relatedness between polypeptide or polynucleotide sequences, as determined by the match between strings of such sequences.

The singular terms “a,” “an,” and “the” include plural referents unless context clearly indicates otherwise. Similarly, the word “or” is intended to include “and” unless the context clearly indicates otherwise. The term “comprises” means “includes.” Also, “comprising A or B” means including A or B, or A and B, unless the context clearly indicates otherwise. Although methods and materials similar or equivalent to those described herein may be used in the practice or testing of this disclosure, suitable methods and materials are described below. In addition, the materials, methods, and examples are illustrative only and not intended to be limiting.

Unless otherwise defined, all technical and scientific terms used herein have the same meaning as commonly understood by one of ordinary skill in the art to which this disclosure belongs. In the case of conflict, the present specification, including definitions, will control. All publications, patent applications, patents, and other references mentioned herein are incorporated by reference. The references cited herein are not admitted to be prior art to the claimed invention.

### HSP Peptides

HSP peptides of this disclosure include substantially pure polypeptides derived from human HSPs. The HSP peptides may be cell-permeable and may inhibit protein aggregation and/or apoptosis.

Embodiments may include one or more peptides derived from Hsp20, one or more peptides derived from αB-crystallin, or a mixture of one or more peptides derived from Hsp20 and αB-crystallin. Peptides derived from additional HSPs may also be utilized, non-limiting examples of which may include αA-crystallin and Hsp27. In some examples, Hsp20 peptides may be the most effective for preventing, reducing, and/or slowing RGC death associated with ocular injury or disease. In additional examples, αB-crystallin peptides may be the most effective for the same or different purposes, such as preventing, reducing, or suppressing retinal endothelial cell death, retinal capillary degeneration, and/or inflammatory cytokine production. Particular HSP peptides may exhibit varying levels of efficacy depending on the subject and/or condition treated and/or the method of treatment.

An Hsp20 peptide may have an amino acid sequence at least about 90%, at least about 95%, or 100% identical to G⁷³HFSVLLDVKHFSPEEIAVK⁹¹ (SEQ ID NO: 1). The Hsp20 peptide may be acetylated in some embodiments. An example of an acetylated Hsp20 peptide may have an amino acid sequence at least about 90%, at least about 95%, or 100% identical to G⁷³HFSVLLDVK(acetyl)HFSPEEIAVK⁹¹ (SEQ ID NO: 2). Intravitreal administration of an Hsp20 peptide similar or identical to SEQ ID NOS: 1 or 2 may be effective to prevent, reduce, and/or slow RGC death in subjects afflicted with glaucoma and/or one or more conditions associated therewith, such as ocular hypertension or axonal degeneration.

An αB-crystallin peptide may have an amino acid sequence at least about 90%, at least about 95%, or 100% identical to ⁷³DRFSVNLDVKHFSPEELKVKV⁹³ (SEQ ID NO: 3). The αB-crystallin peptide may also be acetylated in some embodiments. An αB-crystallin peptide similar or identical to SEQ ID NO: 3 may be effective to prevent, reduce, and/or slow RGC death in subjects afflicted with glaucoma and/or one or more conditions associated therewith, such as ocular hypertension or axonal degeneration. Administration of an αB-crystallin peptide similar or identical to SEQ ID NO: 3 may also be effective to prevent, reduce, and/or slow the death of retinal endothelial cells and/or the irreversible damage of retinal capillary cells, which may be evidenced by an elevated number of acellular capillaries. In some examples, administration of an αB-crystallin peptide similar or identical to SEQ ID NO: 3 may also be effective to reduce or suppress the expression of one or more pro-inflammatory cytokines following ocular injury.

Embodiments may include an HSP peptide conjugated with at least one CPP. The CPP may increase retinal cell penetration of the HSP peptide conjugated therewith, which may increase or maximize the delivery of the HSP peptide to the highest inner limiting membrane of the eye relative to the delivery of the HSP peptide alone. As a result, intravitreal administration of a CPP-conjugated HSP peptide may further prevent, reduce, and/or slow RGC death and/or functional decline associated with ocular injury or disease. In some non-limiting embodiments, a CPP may be conjugated specifically with an αB-crystallin peptide similar or identical to SEQ ID NO: 3. Embodiments may also include a CPP conjugated to one or more Hsp20 peptides, which may have an amino acid sequence similar or identical to SEQ ID NO: 1 or 2. One example of a CPP that may be conjugated with one or more of the disclosed HSP peptides may have an amino acid sequence at least about 80% or 100% identical to VPTLK (SEQ ID NO: 6). Administration of an HSP peptide, e.g., having SEQ ID NO: 2 or 3, conjugated with a CPP having an amino acid sequence at least about 80% or 100% identical to SEQ ID NO: 6 may lead to a particularly significant reduction and/or slowing of RGC death and preserving axonal integrity, which may be driven at least in part by upregulation of one or more components of the CREB signaling pathway.

### Pharmaceutical Compositions

The pharmaceutical compositions of this disclosure are suitable for treating, reducing the risk of, preventing, or alleviating at least one symptom of an ocular disease, injury, and/or condition caused by or associated with retinal cell functional decline and/or retinal cell death, including RGC death, retinal endothelial cell death, and/or retinal capillary degeneration. Embodiments of the pharmaceutical composition may include at least one HSP peptide, which may be conjugated with a CPP. The pharmaceutical composition may also include a pharmaceutically acceptable carrier configured to facilitate and/or stabilize delivery of the HSP peptide to the target site(s) of a subject.

In some embodiments, a pharmaceutical composition may include a mixture of two or more distinct HSP peptides, one or more of which may be conjugated with a CPP. For example, a pharmaceutical composition may include a mixture of Hsp20 peptides and αB-crystallin peptides. According to such examples, the ratio of Hsp20 peptides to αB-crystallin peptides may be about 1:1, about 2:1, about 3:1, about 4:1, about 5:1, about 6:1, about 7:1, about 8:1, about 9:1, about 10:1, about 1:10, about 1:9, about 1:8, about 1:7, about 1:6, about 1:5, about 1:4, about 1:3, or about 1:2.

In embodiments, the pharmaceutical composition may include or be administered concurrently with one or more excipients. Suitable excipients may vary depending upon the particular dosage form utilized. In addition, suitable excipients may be chosen for a particular function, such as the ability to facilitate the production of stable dosage forms. Excipients may also be chosen for regulatory compliance. Non-limiting excipient examples include: fillers, binders, disintegrants, lubricants, glidants, granulating agents, coating agents, wetting agents, solvents, co-solvents, suspending agents, emulsifiers, coloring agents, anticaking agents, humectants, chelating agents, plasticizers, viscosity agents, antioxidants, preservatives, stabilizers, and surfactants. In some embodiments, one or more delivery vehicles may be utilized, non-limiting examples of which may include nanoparticles, nanogels, and/or adeno-associated viral vectors (“AAV vectors”). The skilled artisan will appreciate that certain pharmaceutically acceptable excipients may serve more than one function and may serve alternative functions depending on how much of the excipient is present in the final composition and which other ingredients are present in the composition.

In embodiments, the HSP peptides may be administered concurrently with one or more buffering agents and/or diluents, non-limiting examples of which may include various concentrations of sodium hydroxide and sodium phosphate.

The inclusion of particular excipients and/or carriers may depend on the route of administration. For example, a preparation for parenteral administration may include a sterile aqueous solution, a non-aqueous solvent, a suspension, an emulsion, a freeze-dried preparation, and/or a suppository. Non-aqueous solvents may include propylene glycol, polyethylene glycol, vegetable oil, and/or an injectable ester. As a base for the suppository, witepsol, macrogol, tween 61, cacao butter, laurin butter, glycerogelatin or the like may be used. To increase the stability or absorption of peptides, carbohydrates such as glucose, sucrose or dextran, antioxidants such as ascorbic acid or glutathione, chelating agents, low-molecular weight proteins or other stabilizers may be used.

In some embodiments, the pharmaceutical composition may also be provided as a topical composition, for example in droplet form. Eye drops may be formulated with an aqueous or non-aqueous base also comprising one or more dispersing agents, solubilizing agents, and/or suspension agents. According to such embodiments, the concentration of the HSP peptides in the pharmaceutical composition may be greater than the concentrations utilized for intravitreal implementations.

### Therapeutic Approaches

Methods of treating an ocular condition may involve administering to an eye of a subject a therapeutically effective amount of an HSP peptide composition disclosed herein. The composition may be administered after an ocular injury is sustained, which may include an ocular surgical procedure, or after an ocular disease is diagnosed. Embodiments may also involve administering a disclosed HSP peptide composition to an undiagnosed subject to prevent the subject from developing a disease or to lessen the severity of the symptoms upon disease onset. In some examples, prophylactic administration may be performed after determining that a subject is at an above-average risk of developing an ocular disease or has an above-average risk of sustaining an ocular injury. In some embodiments, intravitreal administration of a disclosed pharmaceutical composition may be uniquely effective at restoring or exceeding natural levels of one or more HSPs in the retinal cells of the subject, including the RGCs (or RGC somas), retinal endothelial cells, and/or retinal capillaries, for instance. Such levels may be sufficient to reduce the retinal cell loss that, if left untreated, would otherwise drive irreversible retinal damage.

An HSP peptide composition may be injected into one or both eyes of a subject soon after and/or during surgery in subjects afflicted with acute angle closure glaucoma. Such treatment may reduce optic nerve degeneration and ultimately slow or prevent complete vision loss. Administration of an HSP peptide composition comprising HSP peptides derived from Hsp20 and/or αB-crystallin may also reduce, prevent, or slow RGC death in subjects diagnosed with primary open angle and normal tension glaucoma. Administration of a composition comprising HSP peptides derived from αB-crystallin, with or without a conjugated CPP, may be particularly effective at reducing, preventing and/or suppressing RGC death, retinal endothelial cell death, retinal capillary degeneration, and/or retinal cell functional decline. In some embodiments, administration of an HSP peptide composition may protect brain neurons after a subject suffers from a traumatic brain injury. The same HSP compositions may also be effective in protecting kidney proximal tubular epithelial cells in diabetic patients.

As noted in the preceding section, the pharmaceutical composition may include, or be administered concurrently with, at least one pharmaceutically acceptable carrier. Relatedly, the pharmaceutical composition may be administered singly or in combination with other therapeutic agents, either serially or simultaneously. Such additional agents may or may not be formulated to treat the same ocular condition(s).

The frequency of administration of the pharmaceutical compositions disclosed herein may vary. In embodiments, a pharmaceutically effective amount of the composition may be administered daily, weekly, monthly, or yearly. The number of times the disclosed compositions are administered to a subject, along with the length of the treatment period, may depend on the severity or type of condition causing, or at risk of causing, retinal damage. For example, embodiments in which the pharmaceutical composition is administered to treat an eye injury may involve fewer discrete administrations than embodiments in which the HSP peptide composition is administered to treat a disease, such as glaucoma, which may require a more sustained treatment approach. The length of the treatment period may also be patient-specific and re-evaluated periodically by a physician or other health care provider. In various embodiments, a pharmaceutical composition may be administered immediately following an injury, such as within one, two, six, 12, or 24 hours after an injury. The formulations may be administered once or multiple times, for example two, three, four, five, six, seven, eight, nine, ten times, or more.

The pharmaceutical compositions disclosed herein may be administered using an injection device, such as tuberculin syringe or an IV drip device, which may be configured specifically for the purposes described herein. In some examples, the administration device may be a single-use device, which may be included in a kit that also includes a single dose of a pharmaceutical composition. Accordingly, an injection device may constitute a part of a system for treating, reducing the risk of, preventing, or alleviating at least one symptom of retinal damage.

While the route of administration may vary, and multiple routes may be acceptable, intravitreal administration of the disclosed compositions may be most effective for treating one or more ocular conditions. Intravitreal administration may be traditionally disfavored due to the potential pain experienced by the subject during and after treatment. Intravitreal administration may also increase the likelihood of bleeding, retinal tears and detachment, cataract formation, and ocular infection. Such potential side effects may be outweighed, however, by the strong efficacy, and reduction in systemic side effects, achieved via localized intravitreal administration of the specific HSP peptide compositions described herein.

Potential alternatives to intravitreal administration may include, topical application on the eye, intraperitoneal injection. After intraperitoneal injection, the disclosed HSP peptide compositions may effectively cross the blood-aqueous barrier and ultimately enter retinal cells, where the HSP peptides of the injected composition may prevent, reduce, or otherwise ameliorate retinal damage. Other non-limiting examples of acceptable administration methods may include intravenous administration, intramuscular administration, subcutaneous administration or local administration.

The therapeutically effective amount of a pharmaceutical composition administered to a subject may vary. In embodiments, each intravitreal dose of a pharmaceutical composition provided to a subject may include an HSP peptide concentration ranging from about 100 μg/mL to about 500 μg/mL. Dosing may depend, for example, on the condition treated, the severity of the condition, the nature of the formulation (e.g., with or without CPP conjugation), the method of administration, the condition of the subject, the age of the subject, the weight of the subject, or combinations thereof. Dosage levels are typically sufficient to achieve a concentration at the site of action that is at least the same as a concentration that has been shown to be active in vitro, in vivo, or in tissue culture.

To accommodate multiple administration techniques and schedules, the pharmaceutical compositions disclosed herein may be prepared in a unit-dosage form or multiple-dosage form, along with a pharmaceutically acceptable carrier and/or excipient according to a method employed by those skilled in the art. Example formulations may be in the form of an aqueous or oil-based solution, a suspension, or an emulsion. For increased stability and long-term storage, the pharmaceutical compositions may be lyophilized.

The following experimental examples are provided to illustrate example embodiments of the present invention, and should not be considered limiting.

### EXAMPLES

### Example 1

To determine the effects of Hsp20 and αB-crystallin peptides against RGC death, a first mouse model of ocular hypertension was adopted in which mice were given intravitreal injections of each peptide.

The first ocular hypertension model was generated by initially anesthetizing mice via intraperitoneal injection of ketamine/xylazine supplemented with a topical application of 0.5% proparacaine hydrochloride. Ocular hypertension was induced unilaterally by injection of polystyrene microbeads (10 μm diameter, 5 million beads/mL of PBS) into the anterior chamber of the right eye of each animal. The cornea was gently punctured near the center using a 33G needle, and a small air bubble was injected to lift the anterior chamber of the eye. A small volume (2 μL) of microbeads was injected into the anterior chamber under the bubble via a micropipette connected to a Hamilton syringe. An antibiotic ointment was applied topically on the injected eye to prevent infection.

The intraocular pressure was measured weekly for six weeks using a tonometer. In particular, the mice were placed in an anesthetic chamber filled with a sustained flow of isoflurane (5% isoflurane at 2 L/minute mixed with oxygen). The tonometer took five measurements for each weekly check-in, eliminated the high and low readings, and generated an average intraocular pressure from the remaining readings for each mouse.

After three weeks of weekly microbead injection to increase the intraocular pressure, a once-weekly, 1-μg dose of either an Hsp20 peptide having SEQ ID NO: 2 (peptain-3a) or an αB-crystallin peptide having SEQ ID NO: 3 (peptain-1) in PBS was also injected intravitreally over a span of three weeks. After six weeks of microbead injection, the eyes were dissected out and post-fixed with 4% PFA overnight at 4° C. The retinas were subsequently dissected out and washed three times in PBS before blocking (5% normal donkey serum and 1% Triton X-100 in PBS) overnight. Whole-mount retinas were then immunostained for Brn3a and βIII-tubulin (1:500 dilution), which are markers for RGCs and axonal cells, respectively. The retinas were then stained with Alexa Fluor 488 donkey anti-mouse or Texas red-conjugated goat anti-rabbit IgG (1:250 dilution). Four image fields from each retinal region were obtained using a 20× lens with a Zeiss confocal. The Brn3a-positive RGC numbers were counted (cells/mm²) in the mid-peripheral regions from four quadrants of the whole-mounted retina using the ImageJ software (NIH). Contralateral uninjured eyes were used as a control.

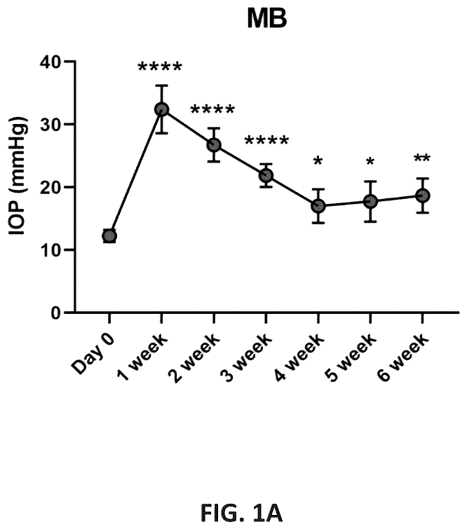

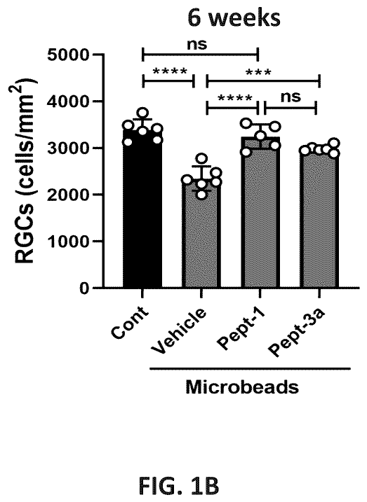

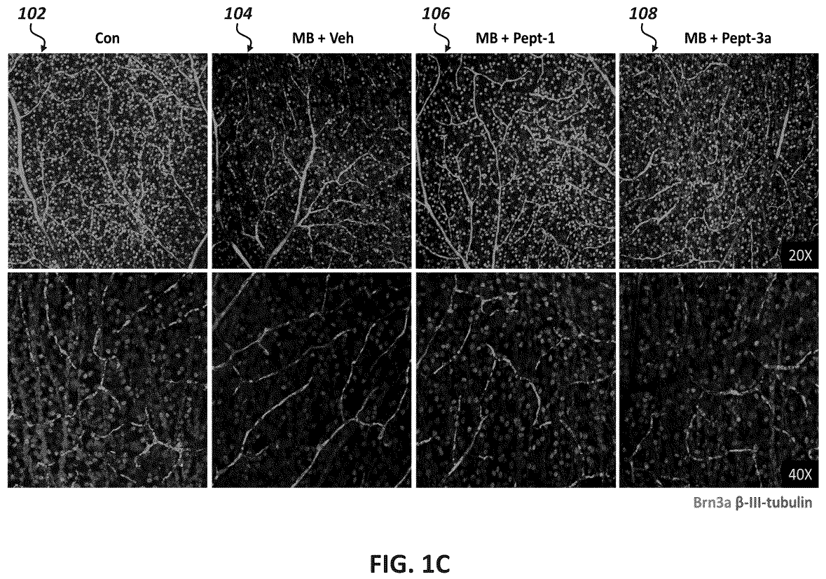

As shown in FIG. 1A, ocular microbead injection elevated the intraocular pressure from 12 mmHg to over 30 mmHg in one week. The intraocular pressure progressively declined thereafter, reaching a low at four weeks. Despite this decrease, intraocular pressures were still significantly higher than in the control mice not injected with microbeads (*p<0.05, **p<0.01, ****p<0.0001 compared to Day 0).

The impact of intravitreal peptide injection on RGC survival is depicted graphically in FIG. 1B, in which ns=not significant, ***p<0.001, ****p<0.0001. As shown, the retinas removed from the control mice (“Cont”) had almost 3,400 RGCs per mm² after six weeks of study participation. By contrast, the untreated mice injected with microbeads and PBS (“Vehicle”) had less than 2,300 RGCs per mm² after six weeks. The retinas extracted from mice treated with peptain-1 (“Pept-1”) had over 3,200 RGCs per mm² after six weeks, and the retinas extracted from mice treated with peptain-3a (Pept-3a″) had about 3,000 RGCs per mm² after six weeks. Accordingly, relative to the vehicle control, both peptain-1 and peptain-3a significantly (and to a similar extent) reduced the loss of RGCs present within retinas having intraocular pressure elevated to hypertension levels.

Confocal microscopy images of the retinas from which the quantitative RGC concentrations of FIG. 1B were obtained are shown in FIG. 1C. As evidenced by greater Brn3a-positive RGC staining, RGC numbers were the highest in the retinas removed from the healthy treatment group 102. Brn3a staining was much lower in the untreated ocular hypertension group 104. The peptain-1 group 106 and peptain-3a group 108 showed a significant reduction in RGC death of 4% and 12% (compared to vehicle control group), respectively. The concurrent βIII-tubulin immunostaining confirmed that both peptain-1 and peptain-3a inhibited axonal degeneration as well, evidenced by the visible reduction in axonal cell death.

### Example 2

To confirm the effects of the same Hsp20 (peptain-3a) and αB-crystallin (peptain-1) peptides against RGC death in mice afflicted with ocular hypertension, a second mouse model of ocular hypertension was developed in which mice were given intravitreal injections of each peptide.

The second ocular hypertension model was generated by anesthetizing mice via intraperitoneal injection of ketamine/xylazine supplemented with topical application of 0.5% proparacaine hydrochloride. A 33G needle was tunneled through the cornea of each tested eye at the superotemporal side close to the limbus to reach the anterior chamber without injuring the lens or iris. Following this hole, about two microliters of silicone oil (1,000 mPa·s) were injected slowly into the anterior chamber until the resulting oil droplet expanded to cover most areas of the iris, and the needle was withdrawn slowly. After the injection, the upper eyelid was gently massaged to close the corneal incision and minimize oil leakage, and veterinary antibiotic ointment was applied to the surface of the injected eye to prevent infection.

After two weeks, the silicone oil droplet was removed. Two corneal tunnel incisions were made using a 33G needle: one tunnel incision superior to the center of the cornea and one tunnel incision inferior to the center of the cornea. A 33G needle attached to a 3 mL Luer lock syringe was used to inject PBS into the anterior chamber of each eye to allow silicone oil outflow through the inferior tunnel incision. Veterinary antibiotic ointment was applied to the surface of the eye after oil removal.

Two days after removal of the oil, a 1-μg dose of either peptain-3a or peptain-1 in PBS was injected intravitreally. The intraocular pressure of each eye was monitored once weekly until four weeks after silicone oil injection using a tonometer under anesthesia with a sustained flow of isoflurane (5% isoflurane at 2 L/minute mixed with oxygen). Two weeks after oil removal (day 28), retinas were removed and immunostained with Brn3a and βIII-tubulin antibodies to visualize and count the surviving RGCs.

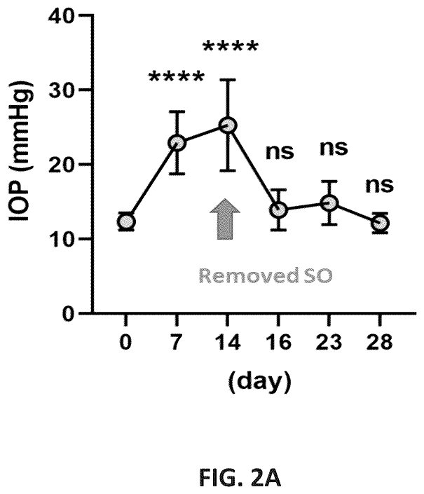

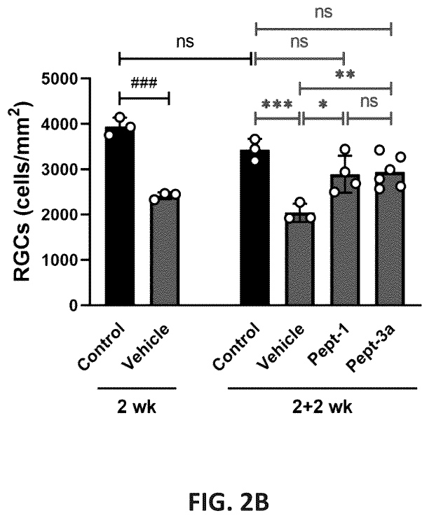

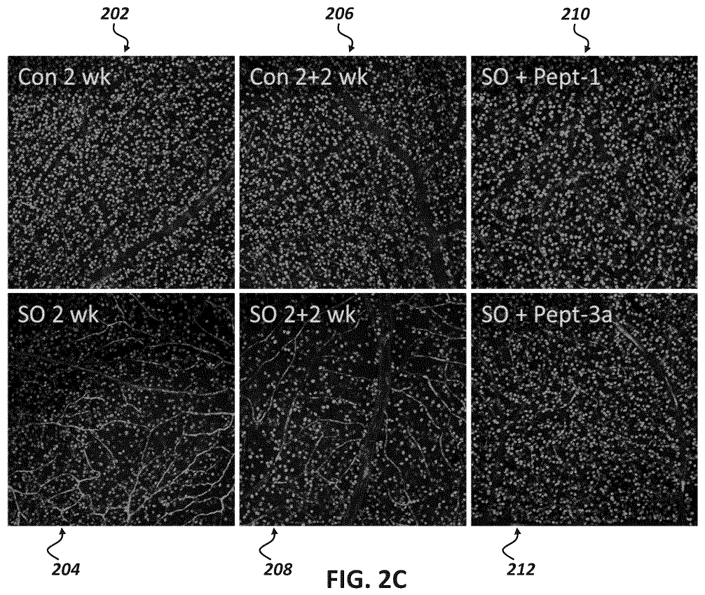

As shown in FIG. 2A, the injected silicone oil (“SO”) caused the intraocular pressure to increase from about 12 mmHg to about 25 mmHg in two weeks. The intraocular pressure dropped to about 13 mmHg two days after oil removal, and stayed within a normal range of about 12 mmHg until day 28.

The impact of intravitreal peptide injection on RGC survival is depicted graphically in FIG. 2B, in which ns=not significant, *p<0.05, **p<0.01, and ***p<0.001 compared to control samples two weeks after silicone oil removal. For the control samples two weeks after silicone oil injection, ### p<0.001.

As shown in the left two bars, the retinas removed from the healthy control mice (not injected with silicone oil) had almost 3,900 RGCs per mm² (“2 wk”), whereas silicone-oil injected retinas contained only 2,400 RGCs per mm² two weeks after silicone oil injection. In addition, the retinas removed from the healthy control mice (not injected with silicone oil) had almost 3,400 RGCs per mm² two weeks after silicone oil removal (“2+2 wk”), whereas the retinas removed from the ocular hypertension mice (“Vehicle”) had only about 2,000 RGCs per mm² two weeks after silicone oil removal. The retinas extracted from mice treated with peptain-1 (“Pept-1”) had just under 2,900 RGCs per mm² two weeks after silicone oil removal (“2+2 wk”), as did the retinas extracted from mice treated with peptain-3a (“Pept-3a”). This data again shows that relative to the vehicle control, both peptain-1 and peptain-3a significantly (and to a similar extent) reduced the loss of RGCs present within retinas having intraocular pressure elevated to hypertension levels.

Confocal microscopy images of the stained RGCs are shown in FIG. 2C. As evidenced by greater Brn3a-positive staining, RGC numbers were the highest in the retinas removed from the healthy control group 202 not injected with silicone oil, and the lowest in the retinas removed from the mice injected with silicone oil (the ocular hypertension group 204) two weeks after injection. Relative to the control group 202, the RGC count decreased by 39% in the untreated ocular hypertension group 204. RGC numbers remained high in the healthy control group 206 at week four, whereas RGC numbers dropped significantly in the untreated ocular hypertension group 208 two weeks after silicone oil removal, falling 41% compared to the PBS-treated group 206 at week four. Peptain-1 treatment 210 and peptain-3a treatment 212 significantly reduced RGC death by 15.7% and 14.2% (compared to PBS-treated control 206), respectively.

### Example 3

A third experiment was conducted to evaluate the ability of peptain-1, specifically, to treat markers of diabetic retinopathy. Because retinal endothelial cell death and retinal capillary cell death are commonly observed in subjects afflicted with diabetic retinopathy, their survival was measured in vitro after pro-inflammatory cytokine-mediated apoptosis accompanied with and without administration of the peptide.

Human retinal endothelial cells (“HRECs”) were treated for three hours with 200 μg/mL of either peptain-1 or a scrambled control peptide in serum-free media. The sequence of the first scrambled control peptide was SLKEKRNFDVSEVKHVLFVDP (SEQ ID NO: 4), and the sequence of the second scrambled control peptide was FEPSVRFSKVDHLVKENDLVK (SEQ ID NO: 5). All test cells were then treated with a combination of pro-inflammatory cytokines (IFN-γ at 50 U/mL+TNF-α at 20 ng/mL+IL-1β at 20 ng/mL) for 48 hours.

Cell lysates were prepared using 1×RIPA buffer containing a protease inhibitor cocktail. Protein concentrations of the cell lysates were measured using the BCA method, and 25 μg of protein were used for immunoblotting. To measure HREC apoptosis, the cells were immunostained for active (cleaved) caspase-3, specifically, which is a marker for apoptosis. β-actin antibody staining was used as a control for measuring levels of a housekeeping protein and for calculating protein density compared to caspase-3 levels. The Western blot membrane treated with cleaved-caspase-3 antibody was eventually stripped and probed for peptain-1 antibody as well.

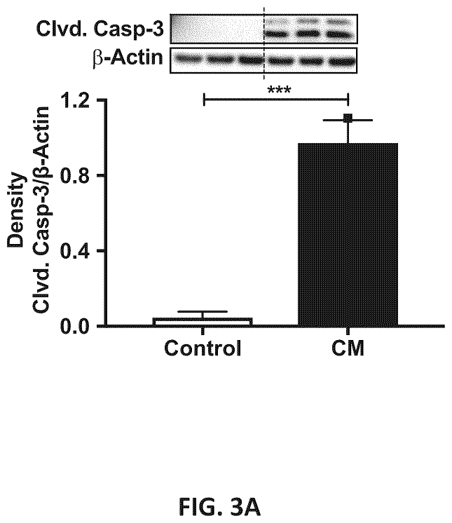

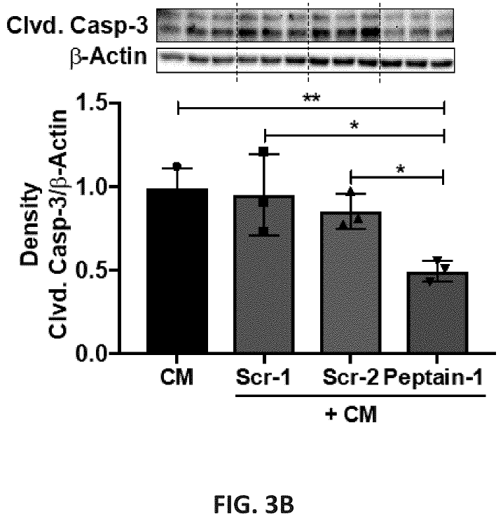

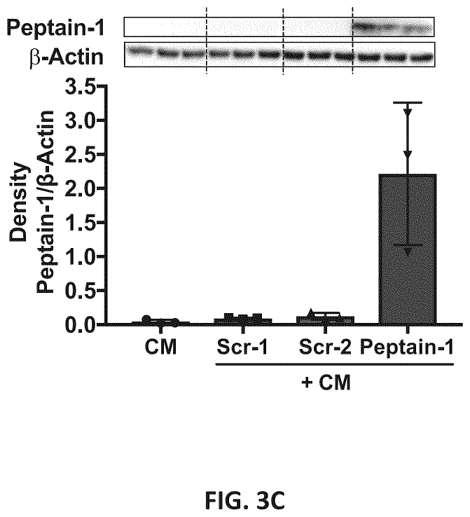

As shown in FIG. 3A, the pro-inflammatory cytokine mixture (“CM”) caused a significant (***p<0.001) increase in HREC apoptosis, as indicated by much greater cleaved caspase-3 levels relative to the control cells not treated with CM (protein levels=mean±SD of triplicate measurements). Levels of β-actin were similar in the control cells and the cells treated with CM, as shown in the Western blot image. The robust levels of cleaved caspase-3 indicative of apoptosis are shown in the Western blot image at the top of the figure, as well as the quantitative bar graph (densitometry plot) beneath it, which depicts the density ratio of measured caspase-3 levels to β-actin levels. As shown, the control cells not treated with CM contained almost no cleaved caspase-3 relative to β-actin, whereas the cells treated with CM included an approximately 1:1 density ratio of caspase-3 to β-actin.

FIG. 3B shows that relative to the control HRECs treated only with CM, the HRECs treated with CM and peptain-1 survived to a significantly greater extent (*p<0.05, **p<0.01), as indicated by the decreased density ratio (˜0.5) of cleaved caspase-3 to β-actin levels for the cells treated with CM and peptain-1 detected by Western blotting, and as shown in the three right-most lanes of the membrane blots, which produced visibly fainter bands for cleaved caspase-3 in cells treated with CM and petpain-1 relative to the cells treated with only with CM and the cells treated with CM and a scrambled peptide (“Scr-1” and “Scr-2”). Relative to the peptain-1 treated cells, significantly more cell death was also observed in the HRECs treated with CM and a scrambled peptide, as shown graphically by the greater density ratio of cleaved caspase-3 to β-actin for such samples (*p<0.05).

FIG. 3C shows that peptain-1 levels were indeed significantly greater in the cells treated with CM and peptain-1. In particular, cells treated with CM and peptain-1 included a significantly greater density ratio (˜2.25) of peptain-1 to β-actin compared to the cells treated only with CM and the cells treated with CM and a scrambled control peptide (“Scr-1” and “Scr-2”). The blot images above the bar graph show an absence of a visible peptain-1 band in the cells treated only with CM and the cells treated with CM and a scrambled peptide.

### Example 4

A fourth experiment was conducted to determine whether peptain-1 can translocate into the retina following intravitreal injection in mice.

To enable visual tracking of the peptide, conjugation of peptain-1 containing a cysteine residue at the C-terminus was performed with sulfo-cyanine5 maleimide (Cy5) dye. Mice (C57BL6/J) were injected intravitreally with 1 μg of peptain-1 conjugated with Cy5 in 2 μL of PBS. The contralateral eyes were used as a negative control. After four hours, Cy5 fluorescence intensity in retinal flat-mounts and homogenized tissue was measured.

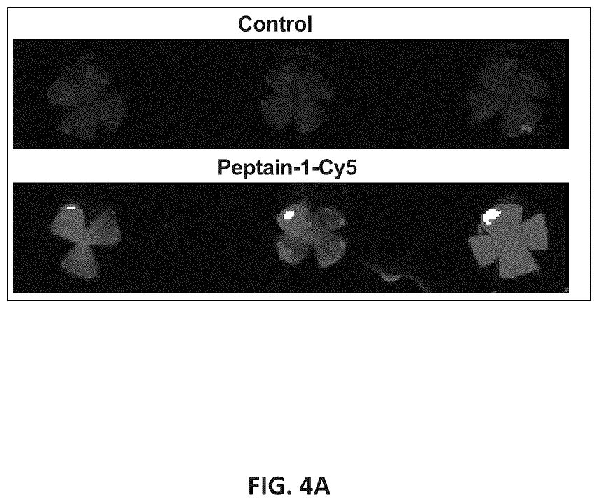

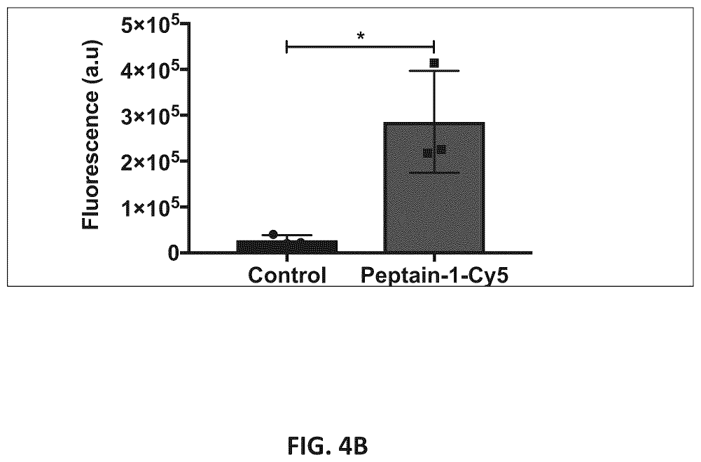

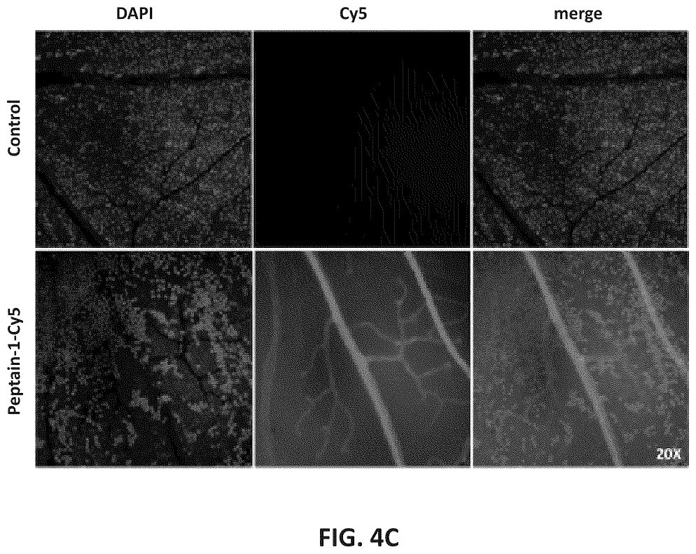

As shown in the retinal flat-mounts of FIG. 4A, Cy5 fluorescence was noticeably greater in the retinas of mice injected with peptain-1-Cy5 relative to the control samples. The fluorescence data shown graphically in FIG. 4B (obtained from homogenized retinal tissue) confirms that peptain-1 did penetrate the retinal tissue of the mice after intravitreal injection without the use of any transfer reagents. Significant retinal blood vessel penetration is shown again in FIG. 4C, after 24 hours of peptain-1-Cy5 injection, in which the first column of images shows retinal blood vessels stained with 4′,6-diamidino-2-phenylindole (“DAPI”), which binds to the DNA present within the nuclei of the retinal cells. As visible in the central image of the top row, no Cy5 fluorescence was detected in the control cells not injected with peptain-1-cy5, whereas in mice injected with peptain-1-cy5 (bottom row, center image), the nuclei of retinal ganglion cells contained significant levels of the peptide. Accordingly, intravitreally injected peptain-1 was distributed throughout the examined retinas about four hours after injection.

### Example 5

A fifth experiment was conducted to determine whether intravitreally injected peptain-1 effectively treats and/or protects retinal capillary cells after ocular injury.

This ocular hypertension model was generated by initially anesthetizing 12-week-old C57BL/6J mice via intraperitoneal injection of ketamine/xylazine supplemented with a topical application of 0.5% proparacaine hydrochloride. To perform the I/R injury, the right eye was cannulated into the anterior chamber with a 33-gauge needle connected to an elevated pouch containing 250 ml of 0.9% NaCl solution. This resulted in an elevation of the intraocular pressure to 120 mmHg for 60 minutes. Peptain-1 or a scrambled peptide (0.5 μg in 1 μL of PBS) were injected intravitreally immediately after I/R injury and again after one week. The PBS-injected eyes were used as vehicle controls. The contralateral eyes were used as additional controls.

Fourteen days after I/R injury or the initial intravitreal injection of peptain-1, mice were sacrificed and enucleated. Retinas were isolated by dissection under light microscopy and treated with elastase (40 U/mL) for 35 minutes at 37° C. with gentle agitation. After careful removal of the internal limiting membrane (ILM), retinas were placed in 12-well plates with Tris-HCl buffer at a pH of 7.8, and then shaken overnight to loosen the remaining RGCs. The retinas were then transferred to glass microscopy slides and the remaining neuronal tissues carefully dislodged through gentle agitation produced by a 20-μL pipette in Tris-HCl. Periodic acid Schiff (PAS) stain was used to visualize the isolated retinal capillary layer. An inverted fluorescence microscope was used to image the mounted capillaries, and the acellular capillaries from each treatment group were counted and analyzed.

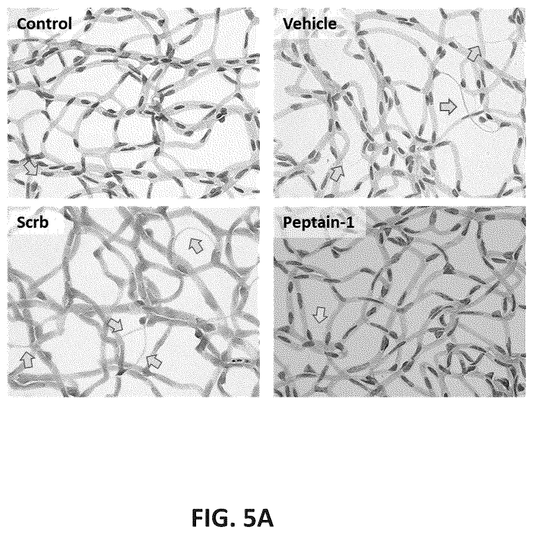

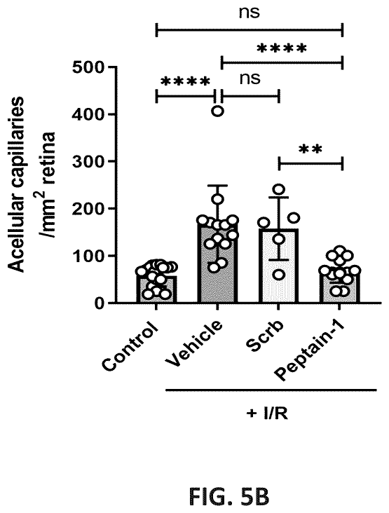

As shown in FIG. 5A, I/R injury (“Vehicle”) significantly increased the number of acellular degenerated retinal capillary cells, which is a common marker for retinal damage, in the vehicle control eyes injected only with PBS relative to retinas not subjected to I/R injury (“Control”). Intravitreally injected scrambled peptide (“Scrb”) showed a similar pattern to the vehicle treatment. Intravitreally injected peptain-1 (“Pept-1”), however, protected retinal cells from I/R injury, evidenced by the lower number of per-retina acellular capillaries present in the peptain-1 -treated cells relative to the untreated cells, as shown in the right-bottom image of the figure, and as represented graphically in the graph of FIG. 5B, in which ns=not significant, **p<0.01, and ****p<0.0001. Intravitreally administered peptain-1 thus reduced the capillary degeneration that would have otherwise occurred naturally after I/R injury.

### Example 6

A sixth experiment was conducted to determine whether peptain-1 (SEQ ID NO: 3) suppresses the production of inflammatory cytokines in the retinas of mice following ocular injury. Mice were subjected to I/R injury, and 0.5 μg peptain-1 or scrambled peptide was intravitreally injected immediately after I/R injury. To measure pro-inflammatory cytokine levels, mice were sacrificed, and retinas were dissected two days after I/R injury. Total RNA was then lysed from the retinas, two micrograms of RNA was reverse-transcribed to synthesize cDNA, and quantitative real-time PCR was performed. The sequences of the PCR primers were as follows: TNF-α forward primer—5′-GACAAGGCTGCCCCGACTA-3′ and reverse primer—AGGGCTCTTGATGGCAGAGA-3′; IL-1β forward primer—5′-5′-GAAATGCCACCTTTTGACAGTG-3′ and reverse primer—5′-TGGATGCTCTCATCAGGACAG-3; IFN-γ forward primer—5′-CAGGCCAGACAGCACTCGAATG-3′ and reverse primer—5′-AGGGAAGCACCAGGTGTCAAGT-3′. The mRNA levels were analyzed using the comparative Ct method (2-ΔΔCT) and then normalized to β-actin (forward primer—5′-AGAAAATCTGGCACCACACC-3′ and reverse primer—5′-GGGGTGTTGAAGGTCTCAAA-3′).

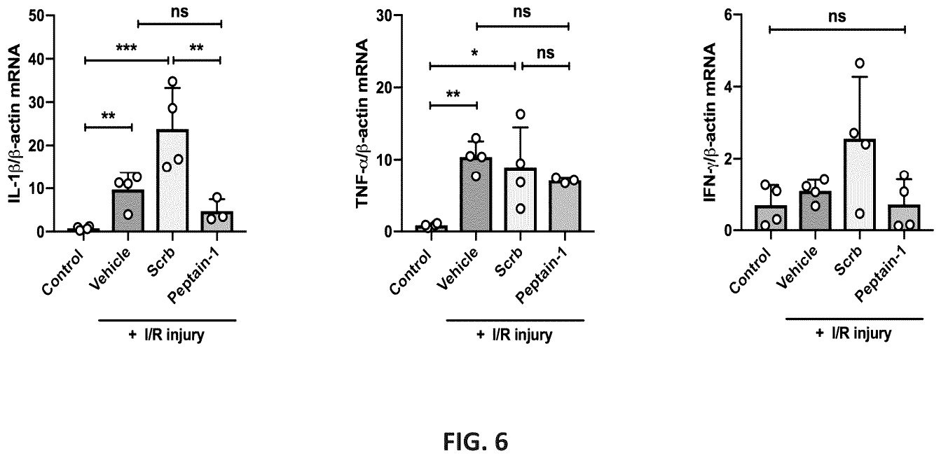

The graphs of FIG. 6 show that at two days after I/R injury, the mRNA levels of IL-1β and TNF-α were significantly increased (by 12.6-fold and 12.0-fold, respectively) in the vehicle control group (“Vehicle”) compared to contralateral retinas (“Control”), and the scrambled peptide-treated group showed a similar pattern to the vehicle group after I/R injury (30.9-fold and 10.4-fold increase, respectively) (Scrb=scrambled peptide, ns=not significant, *p<0.05, **p<0.01, and ***p<0.001, n=3-4). The mRNA level of IFN-γ was not significantly changed in any group. Peptain-1 treatment reduced the increase in IL-1β expression by 4.6-fold and TNF-α expression by 6.2-fold compared to the vehicle group after I/R injury. Accordingly, intravitreally injected peptain-1 reduced the pro-inflammatory cytokine upregulation in retinal cells that would have otherwise occurred naturally after I/R injury. Without being bound to any particular theory, peptain-1 may therefore intercept one or more inflammatory pathways that contribute to retinal capillary degeneration after ocular injury.

### Example 7

A seventh experiment was conducted to determine whether peptain-1 (P1) levels in HRECs were greater after incubating the cells for 20 hours with P1 conjugated to a CPP (P1-CPP) relative to a 20-hour incubation with P1 only.

HREC lysates were prepared using 1×RIPA buffer containing a protease inhibitor cocktail. Protein concentrations of the cell lysates were measured using the BCA method, and 25 μg of protein were used for immunoblotting. P1 levels in cultured HRECs were measured using an anti-P1 antibody after an incubation period. β-actin antibody staining was used as a control for measuring levels of a housekeeping protein and for calculating P1/β-actin density levels.

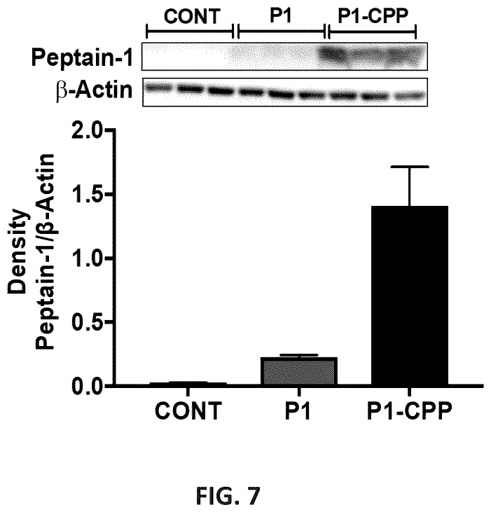

As shown in the Western blot image of FIG. 7, levels of β-actin in the control cells not treated with P1 or P1-CPP were similar to the levels measured in cells incubated with either P1 or P1-CPP. The level of P1 detected within the cells incubated with P1 was greater than in the control cells, as measured by the density of P1 to β-actin. The level of P1 detected in cells treated with P1-CPP, by contrast, was significantly greater than the level of P1 detected in the P1-only cells, as indicated by the significantly greater density ratio of P1 to β-actin compared to the cells incubated only with P1 (P1 vs. P1-CPP ***p<0.001). CPP thus effectively increased the penetration and retention of P1 in HRECs over a 20-hour period.

### Example 8

An eighth experiment was conducted to determine whether P1-CPP can inhibit RGC death in a rodent model of glaucoma generated by trophic factor deprivation.

Primary RGCs were first isolated from post-natal rat pups ranging from four to six days old. A treatment group of isolated RGCs was then cultured and deprived of trophic factors for 48 hours in the presence of 12.5 μg/mL P1-CPP. Control cells (treated with a vehicle) were separately cultured and deprived of trophic factors for 48 hours, but without of the addition of P1-CPP. RGC survival in both groups was then assessed by monitoring the cells via CytoCalcein™ Violet 450 fluorescence. The experiment was conducted four times for statistical significance.

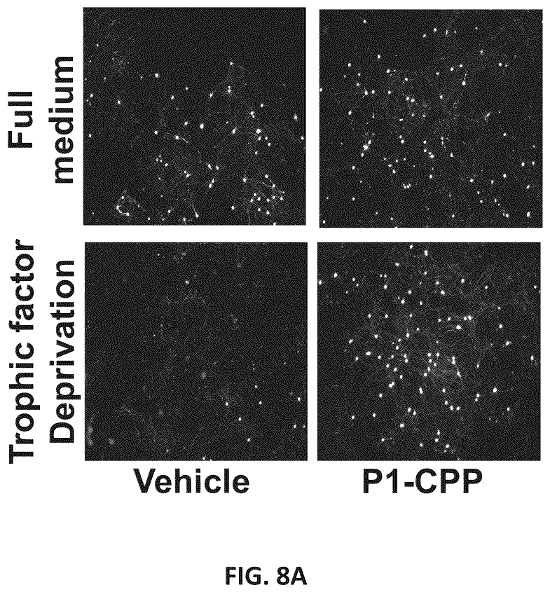

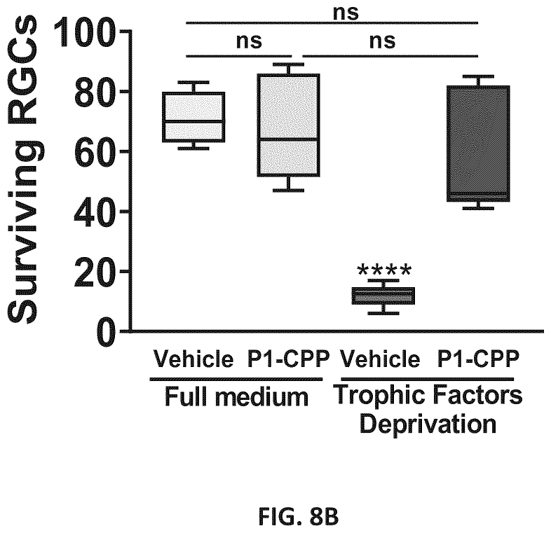

As shown in the microscopy images of FIG. 8A and confirmed in the quantitative bar graph of FIG. 8B, a similar number of control RGCs and RGCs treated with P1-CPP survived in full medium containing standard trophic factor levels (brain-derived neurotrophic factor ((BDNF, 50 ng/mL; Peprotech, Rocky Hill, NJ, USA)), ciliary neurotrophic factor (CNTF, 10 ng/mL; Peprotech), and forskolin (5 ng/mL; Sigma-Aldrich Corp., St. Louis, MO, USA). P1-CPP treatment significantly lowered the neurotrophic factor-mediated RGC loss by 64% (****p<0.0001; ns=not significant). Accordingly, P1-CPP protected rat RGCs from death typically induced by trophic factor deprivation.

### Example 9

A ninth experiment was conducted to determine whether P1-CPP can inhibit RGC loss in a rodent model of glaucoma generated by endothelin-3-mediated death.

Primary RGCs were isolated from post-natal rat pups ranging from four to six days old. Primary RGCs were treated with endothelin-3 (ET-3; 100 nM) in the presence of either P1-CPP (12.5 μg/mL) or vehicle, following which RGC survival was assessed by monitoring CytoCalcein™ Violet 450 (Invitrogen), which is a membrane-permeable live-cell labeling dye. The experiment was conducted twice for statistical significance.

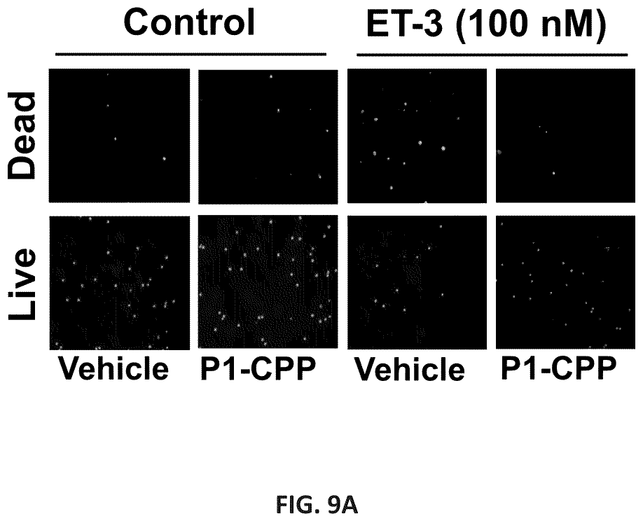

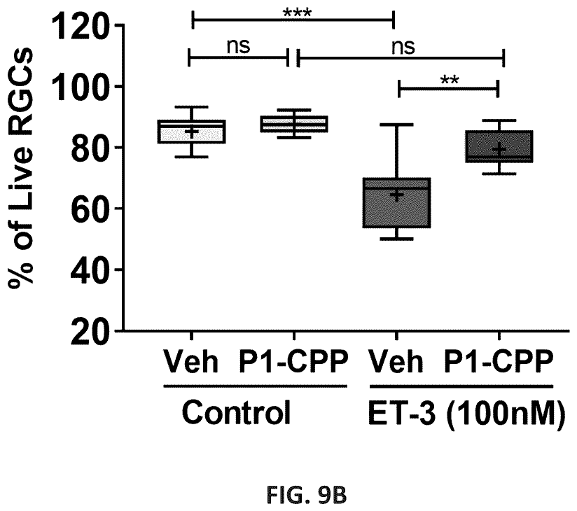

As shown in the fluorescence images of FIG. 9A and corresponding bar graph of FIG. 9B, endothelin-3 application significantly reduced RGC survival in the cells (64% survival) not treated with P1-CPP (p***<0.001). As further shown, a greater percentage of endothelin-3-treated RGCs survived in the presence of P1-CPP (79.5% survival) than in the presence of a vehicle control (**p<0.01). The percentage of surviving RGCs was in fact similar in the P1-CPP-treated cells subjected to endothelin-3 relative to the control cells not subjected to endothelin-3 or P1-CPP.

Accordingly, P1-CPP effectively protected rat RGCs from edothelin-3-induced death.

### Example 10

A tenth experiment was conducted to determine whether intravitreal administration of P1-CPP can inhibit RGC loss in a rodent model of glaucoma generated by elevated intraocular pressure (Morrison's glaucoma model).

To elevate intraocular pressure (TOP), approximately 50-100 μL of 1.8 M NaCl was injected into an episcleral vein of the rat's eyes with a force sufficient to blanch the aqueous plexus. This procedure produces scarring of the trabecular meshwork with a resultant rise in IOP and damage to the optic nerve. IOP-elevated eyes were then intravitreally injected with 2 μg (per eye) of either P1-CPP or a vehicle control on a weekly basis for a total of six weeks. Untreated naïve rats were used as a negative control. After six weeks, the eyes were dissected out and fixed with 4% PFA overnight at 4° C. The retinas were then incubated with the primary antibody, goat anti-Brn3a (1:200, SC-31984, Santa Cruz Biotechnology, Inc.) for three days at 4° C. After three washes with PBS, the retinas were incubated overnight in the corresponding secondary antibodies: Alexa 488 conjugated donkey anti-goat antibody (1:1000 dilution, A11055, Invitrogen) at 4° C. After washes, cuts were made in the four quadrants (superior, inferior, nasal and temporal) and retinal flat mounts were prepared and mounted using Prolong Gold anti-fade (Life Technologies). The experiment was conducted three times for statistical significance. Images of flat mounts were captured using magnification ×20 in a Cytation5 microscope. Images were taken at two different eccentricities, located at one third (mid-peripheral) and two thirds (peripheral) of the distance between the optic nerve head to the periphery of retina. Two images were captured at each eccentricity, in each of the four quadrants, including the superior, inferior, nasal, and temporal quadrants, for a total of 16 images per retina. RGC counts were determined by manual counting. The counting was performed by a masked observer who was unaware of the treatment groups of the animals.

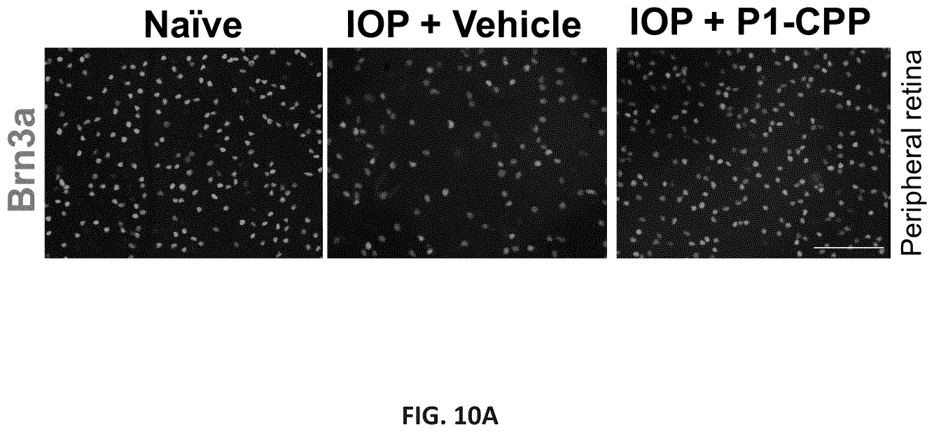

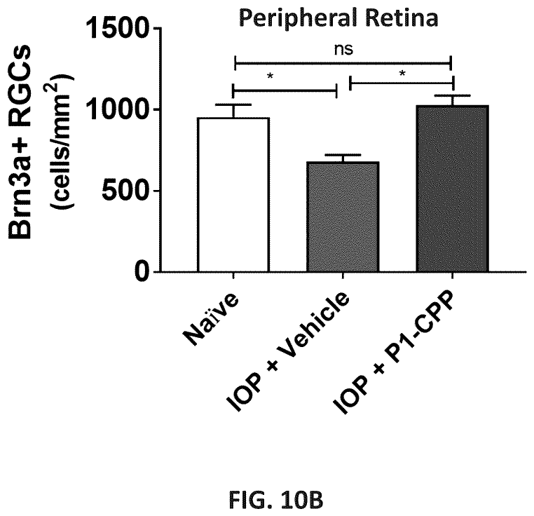

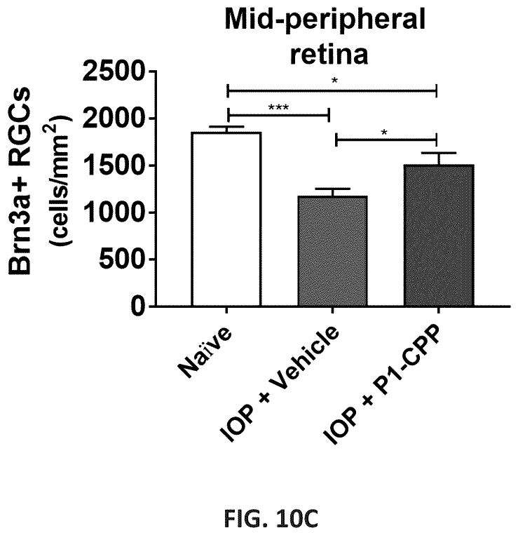

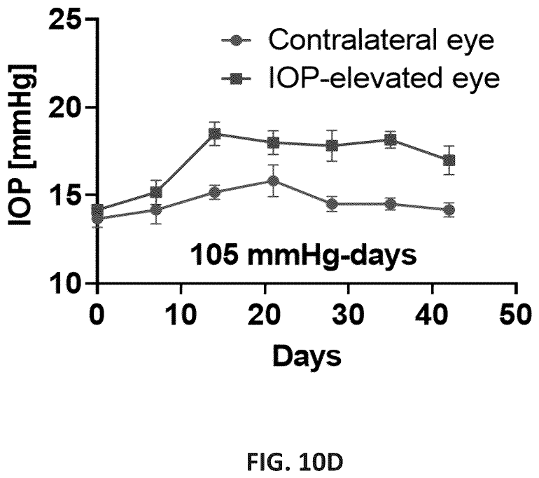

As shown in the fluorescence images of FIG. 10A and corresponding bar graph of FIG. 10B, a greater number of pressure-elevated (“TOP”) peripheral retinal ganglion cells survived after P1-CPP application than pressure-elevated vehicle-injected control cells (*p<0.03; ns=not significant). Results were similar in mid-peripheral retinal cells, as shown in FIG. 10C (***p<0.001), indicating that P1-CPP treatment enhanced RGC survival in rodent eyes subjected to elevated intraocular pressure. FIG. 10D confirms that the intraocular pressure, measured in mmHg, was indeed elevated significantly in the targeted eye of each rat over the six-week duration of the trial relative to the contralateral controls.

### Example 11

An eleventh experiment was conducted to determine whether intravitreal administration of P1-CPP can promote optic nerve axon protection in a rodent model of glaucoma generated by elevated intraocular pressure (Morrison's glaucoma model).

As in Example 10, intraocular pressure was elevated in the eyes of Brown Norway rats, which were then intravitreally injected with 2 μg (per eye) of either P1-CPP or a vehicle control on a weekly basis for a total of six weeks. Untreated naïve rats were used as a negative control. The optic nerves were fixed with 2% paraformaldehyde, 2.5% glutaraldehyde in 0.1 M sodium cacodylate buffer. Before dehydration, the optic nerves were transferred to 2% osmium tetroxide in PBS for one hour and embedded in Epon. Optic nerve cross sections were obtained using the ultramicrotome and stained with 1% PPD, and images of the stained sections were taken in a Zeiss LSM 510 META confocal microscope using an oil immersion magnification ×100. Images were taken at five points covering the center, as well as the peripheral region of each quadrant of every optic nerve section.

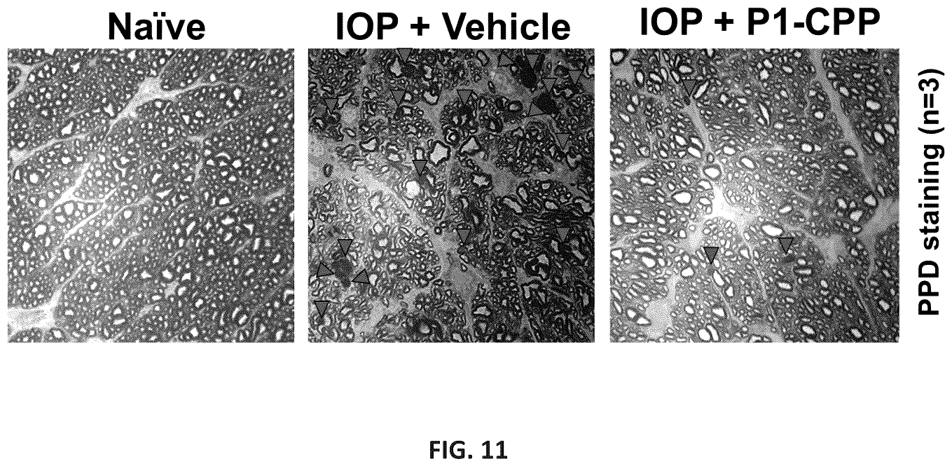

The microscopy images of FIG. 11 show that of the retinal ganglion cells subjected to elevated intraocular pressure, a greater number of collapsed axons (indicated by the arrows) were observed following treatment with a vehicle control relative to treatment with P1-CPP. Accordingly, P1-CPP promoted optic nerve axon protection in a rat glaucoma model generated by elevating intraocular pressure.

### Example 12

A twelfth experiment was conducted to determine whether intravitreal administration of P1-CPP can alleviate RGC function decline in a rodent model of glaucoma generated by elevated intraocular pressure (Morrison's glaucoma model in rats).

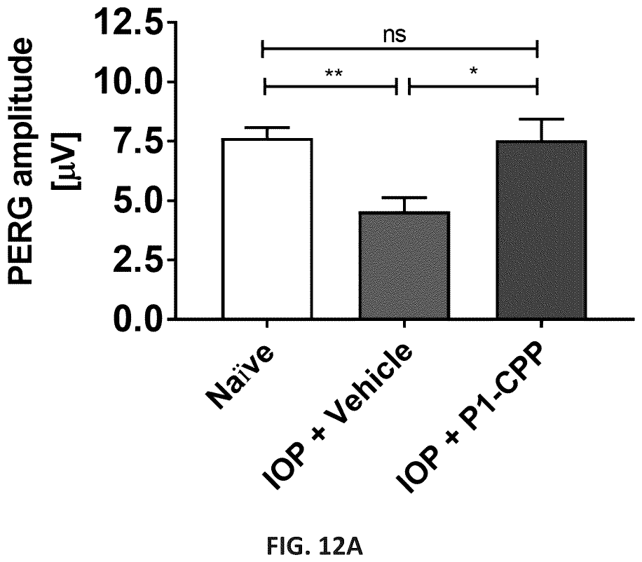

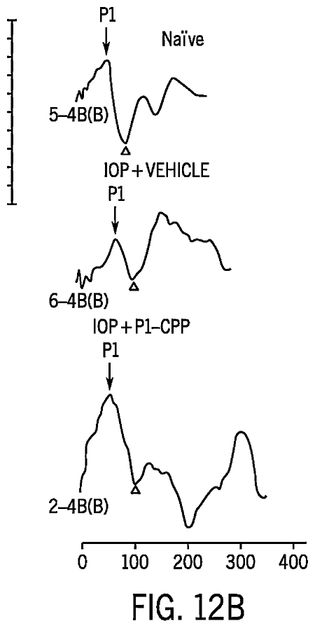

As in Examples 10 and 11, intraocular pressure was elevated in the eyes of Brown Norway rats, which were then intravitreally injected with 2 μg (per eye) of either P1-CPP or a vehicle control on a weekly basis for a total of six weeks. Untreated naïve rats were used as a negative control, and RGC function was assessed via pattern electroretinogram (PERG) amplitude measured at the 6-week mark following IOP-elevation surgery. Briefly, rats were anesthetized by intraperitoneal injection (100 μL/100 g body wt) of a ketamine (VEDCO, Saint Joseph, MO)/xylazine (VEDCO, Saint Joseph, MO) cocktail with final concentrations of 55.6 mg/mL/5.6 mg/mL, respectively. PERG measurements were conducted using the Jörvec instrument (Intelligent hearing systems, Miami, FL) Miami PERG system (Jörvec, Miami, FL), as per manufacturer's instructions. Reference and ground electrodes were placed subcutaneously in the scalp and at the tail region, respectively, and corneal electrodes were positioned at the lower fornix in contact with the eye globe. Small drops of GelTear eye drops were applied to both eyes to prevent corneal dryness. Two separate LED monitors attached to the system were used to display contrast-reversing horizontal bars at a spatial frequency of 0.095 cycles/degree and luminance of 500 cd/m2. The distance between the display monitors and the eyes were maintained at 10 cm. The LED monitors were placed at an angle of approximately 60 degrees for a better projection of the light signals. PERG waveforms generated for each run consisting of 372 sweeps (on-off) from both eyes were then processed and averaged by the PERG software separately for each eye. The grand average PERG waveforms were analyzed using the PERG software to identify the major positive (P1) and negative waves (N2) to calculate the amplitude and latency. The PERG amplitude readings (measured in μV) are shown in FIG. 12A. IOP elevation caused a 63% decline in the PERG amplitude (4.55 μV) compared to naïve rats (7.65 μV), which was sustained by P1-CPP treatment (7.55 μV). (**p<0.001; *p<0.03; ns=not significant). The corresponding PERG traces are shown in FIG. 12B. Accordingly, P1-CPP treatment improved the visual function of RGCs in rat glaucoma models generated by elevating intraocular pressure.

Examples 7-12 indicate that P1-CPP may not only improve retinal cell penetration of P1 compared to P1 alone, but may also protect primary rat RGCs from neurotrophic factor deprivation and endothelin-3-induced cell death. Intravitreal administration of P1-CPP can also protect RGC axons from collapsing during a six-week period of intraocular pressure elevation, and can provide functional protection for RGCs subjected to the same conditions. Together, these results suggest that P1-CPP may be developed as a neuroprotective agent for treating glaucoma in humans.

### Example 13

A thirteenth experiment was conducted to determine whether P1-CPP can inhibit RGC loss in a human retinal explant model of RGC death generated via neurotrophic factor deprivation.

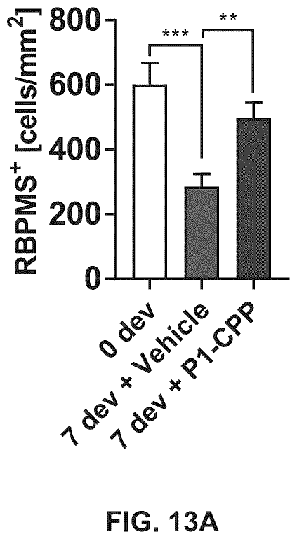

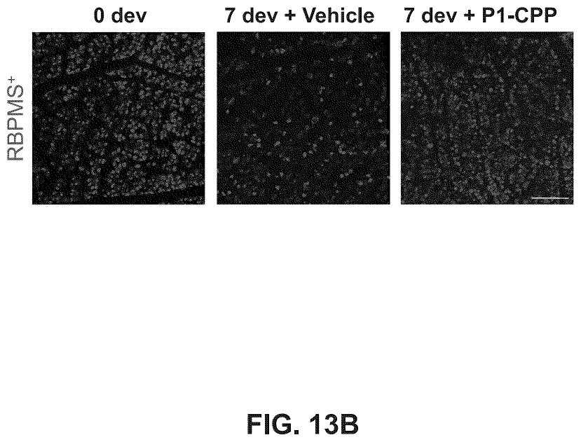

Ex vivo human retinal explants (n=3 donors) were obtained within 12 hours postmortem, subjected to neurotrophic factor deprivation, and incubated with either 12.5 μg/mL P1-CPP or a vehicle control for 7 days (7 dev). Retinal explants not subjected to neurotrophic factor deprivation were used as a control (0 dev). Explants were then stained with RBPMS, which is an RGC marker, and the surviving RGCs were counted using ImageJ. As shown by the greater number of RBPMS⁺ cells represented in the bar graph of FIG. 13A and visible in the confocal microscopy image of FIG. 13B, 7 days of P1-CPP treatment applied to the RGCs subjected to neurotrophic factor deprivation significantly (p<0.005) reduced RGC loss in the explants relative to the vehicle control cells. These results show that P1-CPP treatment decreases neurotrophic factor deprivation-mediated RGC loss in human retinal explants.

### Example 14

A fourteenth experiment was performed to identify the molecular pathways impacted by P1-CPP treatment in a rodent model of glaucoma generated by elevated intraocular pressure (Morrison's glaucoma model in rats).

Intraocular pressure was elevated in the eyes of Brown Norway rats, which were then intravitreally injected with 2 μg (per eye) of either P1-CPP or a vehicle control once a week for a period of 2 weeks. Untreated naïve rats were used as a negative control. Rats were euthanized and primary adult RGCs were isolated by the modified immunopanning method. Total RNA was isolated using the Trizol/column method, and RNA-sequencing was performed using an Illumina platform. The resulting FASTQ files were uploaded into Galaxy for analysis with FASTQC, RNASTAR, feature counts, and DESeq2. The results from DESeq2 were then assessed using Qiagen's Ingenuity Pathway Analysis (IPA) to identify significantly upregulated and downregulated pathways.

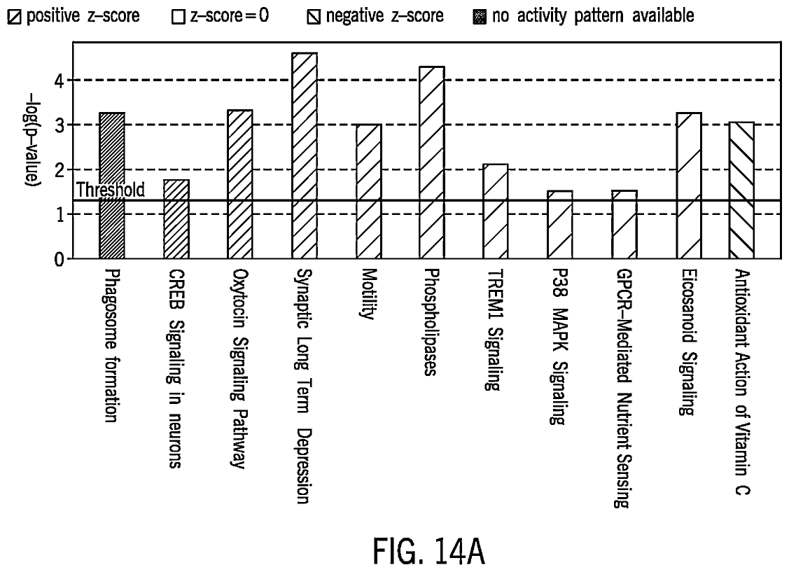

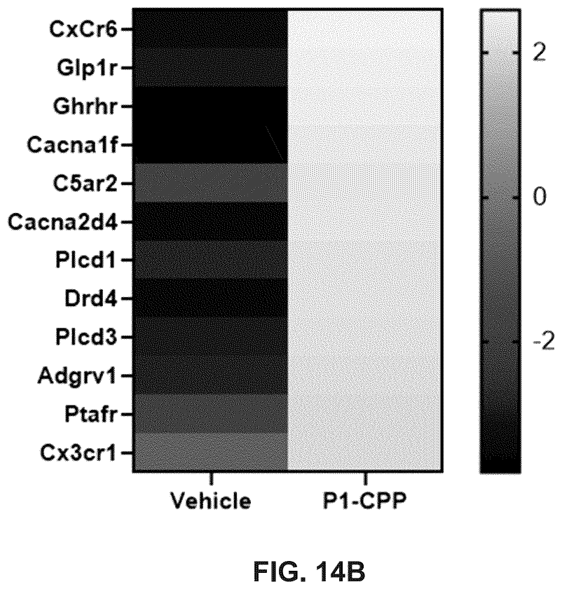

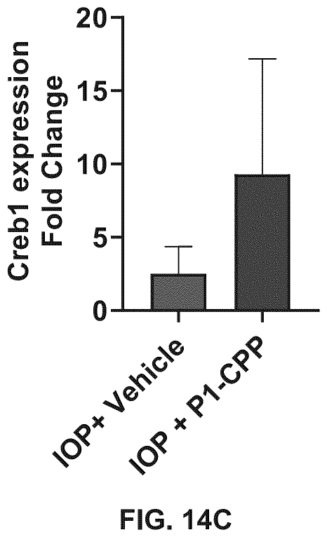

RNA sequencing analysis of rat RGCs isolated following 2 weeks of IOP elevation revealed that RGCs treated with P1-CPP had several differentially expressed pathways compared to vehicle-treated groups, including 6,343 significantly upregulated and 5,960 significantly downregulated genes. Pathways and molecular processes significantly upregulated following P1-CPP treatment included: phagosome formation, CREB signaling in neurons, oxytocin signaling, synaptic long-term depression, motility, phospholipases, TREM1 signaling, p38 MAPK signaling, GPCR-sensing and eicosanoid signaling (FIG. 14A). Notably, the TOP- and vehicle-treated RGCs, when compared to the naïve group, demonstrated decreased expression of multiple components of the CREB signaling pathway, including Creb-1, c-RAF, MEK1/2, ERK1/2, and p90RSK. This decline was prevented by P1-CPP treatment, as delineated in the heat map of the top 12 differentially expressed genes in the CREB pathway shown in FIG. 14B. Quantitative PCR further confirmed the results obtained by RNA sequencing, showing the increased expression of Creb-1 in P1-CPP-treated rats compared to Creb-1 expression in the vehicle-treated group (FIG. 14C).

In view of these data, potential mechanisms of action of P1-CPP in treating glaucoma modeled in rodents include activation of the pro-survival CREB signaling pathway, phagosome formation, and long-term synaptic depression to prevent cell death and vision loss.

Although various representative embodiments and implementations have been described above with a certain degree of particularity, those skilled in the art could make numerous alterations to the disclosed embodiments without departing from the spirit or scope of the inventive subject matter set forth in the specification and claims. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present disclosure. It is intended that all matter contained in the above description or shown in the accompanying drawings shall be interpreted as illustrative only and not limiting. Changes in detail or structure may be made without departing from the spirit of the disclosure as defined in the appended claims.

1. A method of treating, reducing the risk of, preventing, or alleviating at least one symptom of a retinal disease, injury, or condition in a subject, the method comprising:
administering intravitreally to an eye of the subject a therapeutically effective amount of a composition comprising at least one polypeptide derived from a biologically active heat shock protein,
wherein the heat shock protein comprises Hsp20, and
wherein the at least one polypeptide is conjugated with a cell-penetrating peptide.

2. The method of claim 1, wherein the cell-penetrating peptide has an amino acid sequence at least 80% identical to VPTLK.

3. The method of claim 1, wherein the at least one polypeptide has an amino acid sequence at least 90% identical to G⁷³HFSVLLDVK(acetyl)HFSPEEIAVK⁹¹.

4. The method of claim 1, wherein the at least one polypeptide has an amino acid sequence at least 90% identical to ⁷³DRFSVNLDVKHFSPEELKVKV⁹³.

5. The method of claim 1, wherein the composition is administered during or after an ocular surgical procedure.

6. The method of claim 1, wherein the retinal disease, injury, or condition is glaucoma.

7. The method of claim 1, wherein the retinal disease, injury, or condition comprises: macular degeneration, diabetic retinopathy, retinal detachment, or retinitis pigmentosa.

8. The method of claim 1, wherein the retinal disease, injury, or condition is caused by excitotoxic damage, physical damage, chemical damage, neurotrophic factor deprivation, oxidative stress, inflammation, mitochondrial dysfunction, axonal transport failure, or combinations thereof.

9. The method of claim 1, wherein the retinal disease, injury, or condition comprises: a loss of human retinal ganglion cells, ocular hypertension, optic nerve degeneration, pathological apoptosis, or protein aggregation.

10. A system for treating, reducing the risk of, preventing, or alleviating at least one symptom of a retinal disease, injury, or condition in a subject, the system comprising:
a therapeutically effective amount of a composition comprising at least one polypeptide derived from a biologically active heat shock protein,
wherein the heat shock protein comprises Hsp20,
wherein the at least one polypeptide is conjugated with a cell-penetrating peptide; and
an intravitreal injection device configured to administer the composition to the subject.

11. The system of claim 10, wherein the cell-penetrating peptide has an amino acid sequence at least 80% identical to VPTLK.

12. The system of claim 10, wherein the at least one polypeptide has an amino acid sequence at least 90% identical to G⁷³HFSVLLDVK(acetyl)HFSPEEIAVK⁹¹.

13. The system of claim 10, wherein the at least one polypeptide has an amino acid sequence at least 90% identical to ⁷³DRFSVNLDVKHFSPEELKVKV⁹³.

14. The system of claim 10, wherein the retinal disease, injury, or condition is glaucoma.

15. The system of claim 10, wherein the retinal disease, injury, or condition comprises: macular degeneration, diabetic retinopathy, retinal detachment, or retinitis pigmentosa.

16. The system of claim 10, wherein the retinal disease, injury, or condition comprises: retinal ganglion cell loss, retinal endothelial cell loss, retinal capillary cell loss, ocular hypertension, optic nerve degeneration, pathological apoptosis, and protein aggregation.

17. A pharmaceutical composition comprising:
at least one polypeptide derived from Hsp20, wherein the at least one polypeptide is conjugated with a cell-penetrating peptide; and
a pharmaceutically acceptable carrier,
wherein the pharmaceutical composition is formulated for treating, reducing the risk of, preventing, or alleviating at least one symptom of a retinal disease, injury, or condition in a subject, and
wherein the pharmaceutical composition is formulated for intravitreal administration.

18. The pharmaceutical composition of claim 17, wherein the cell-penetrating peptide has an amino acid sequence at least 80% identical to VPTLK.

19. The pharmaceutical composition of claim 17, wherein the at least one polypeptide has an amino acid sequence at least 90% identical to G⁷³HFSVLLDVK(acetyl)HFSPEEIAVK⁹¹ or ⁷³DRFSVNLDVKHFSPEELKVKV⁹³.

20. The pharmaceutical composition of claim 17, wherein the retinal disease, injury, or condition is glaucoma.
